(19) United States
(12) Patent Application Publication
(10) Pub. No.: US 2013/0184290 A1
(43) Pub. Date: Jul. 18, 2013

(54) SELF-EMULSIFYING FORMULATIONS AND METHODS OF USE THEREOF

(71) Applicants: Zalicus Pharmaceuticals Ltd., Vancouver (CA); CAPSUGEL, Inc., Morristown, NJ (US)

(72) Inventors: Mahesh V. PADVAL, Needham, MA (US); Abizer HARIANAWALA, Acton, MA (US); Elizabeth GRIECO, Reading, MA (US); Vishal SAXENA, Overland Park, KS (US)

(73) Assignees: CAPSUGEL, Inc., Morristown, NJ (US); Zalicus Pharmaceuticals Ltd., Vancouver (CA)

(21) Appl. No.: 13/661,729

(22) Filed: Oct. 26, 2012

Related U.S. Application Data
(63) Continuation of application No. 13/414,390, filed on Mar. 7, 2012, now abandoned.
(60) Provisional application No. 61/450,469, filed on Mar. 8, 2011.

(51) Int. Cl. A61K 31/495

(52) U.S. Cl. CPC A61K 31/495
USPC 514/255.01; 435/375

(57) ABSTRACT

The present invention relates to formulations and methods for increasing the bioavailability of 1-(4-benzhydrylpiperazin-1-yl)-3,3-diphenylpropan-1-one, diphenylpropanoyl)piperazine, or a salt thereof. In particular, the formulation can include one or more self-emulsifying carriers.

### CROSS REFERENCE TO RELATED APPLICATION

This application is a continuation of U.S. application Ser. No. 13/414,390, filed on Mar. 7, 2012, which claims benefit of U.S. Provisional Application No. 61/450,469, filed on Mar. 8, 2011, each of which is hereby incorporated by reference in its entirety.

### BACKGROUND OF THE INVENTION

The present invention relates to formulations and methods for increasing the bioavailability of 1-(4-benzhydrylpiperazin-1-yl)-3,3-diphenylpropan-1-one or a metabolite thereof, as well as salts thereof. In particular, the formulations and methods relate to the use of self-emulsifying carriers to improve mean bioavailability in fasted subjects and/or to reduce food effect.

Ion channels mediate a variety of normal physiological functions and are also implicated in a number of human disorders. Examples of human disorders mediated by calcium channels include but are not limited to congenital migraine, cerebellar ataxia, angina, epilepsy, hypertension, ischemia, and some arrhythmias (see, e.g., Janis et al., Ion Calcium Channels: Their Properties, Functions, Regulation and Clinical Relevance (1991) CRC Press, London); and those mediated by sodium channels include but are not limited to epilepsy, cancer, pain, migraine, Parkinson's Disease, mood disorders, schizophrenia, psychosis, tinnitus, amyotrophic lateral sclerosis, glaucoma, ischaemia, spasticity disorders, obsessive compulsive disorder, restless leg syndrome, and Tourette syndrome. Modulators of ion channels, e.g., such as 1-(4-benzhydrylpiperazin-1-yl)-3,3-diphenylpropan-1-one, a metabolite thereof, or a salt thereof, are thus desired. In particular, formulations of such modulators having improved oral bioavailability and/or reduced patient-to-patient variability in pharmacokinetic behavior are needed.

### SUMMARY OF THE INVENTION

The invention provides formulations and methods for administering 1-(4-benzhydrylpiperazin-1-yl)-3,3-diphenylpropan-1-one (compound 1), a metabolite thereof (e.g., 1-(3,3-diphenylpropanoyl)piperazine (compound 2)), or a salt thereof. Compound 1 is a potent N-type calcium channel antagonist having selectivity over other types of calcium channels (e.g., L-type or P/Q-type calcium channels). The invention also provides use of these formulations for acting at ion channels (e.g., calcium channels (e.g., N-type calcium channels) and/or sodium channels) and for treating various conditions associated with these channels, such as pain and epilepsy.

In a first aspect, the invention relates to a pharmaceutical composition in unit dosage form for oral administration, the composition including from about 20 mg to about 250 mg of compound 1, compound 2, or a salt thereof, and a pharmaceutically acceptable carrier, where, following administration of the pharmaceutical composition to subjects, the ratio of the mean bioavailability for fed subjects to the mean bioavailability for fasted subjects is from about 1.0 to about 2.0.

In a second aspect, the invention relates to a pharmaceutical composition in unit dosage form for oral administration, the composition including from about 20 mg to about 250 mg of compound 1, compound 2, or a salt thereof, and a pharmaceutically acceptable carrier (e.g., a self-emulsifying pharmaceutically acceptable carrier).

In some embodiments of any of the aspects herein, the composition includes from about 20 mg to about 250 mg of compound 1, compound 2, or a salt thereof, in a carrier that, together with the compound 1, compound 2, or the salt thereof, is self-emulsifying.

In some embodiments, the composition includes from about 2% to about 10% (w/w) of compound 1 or compound 2 (e.g., from 2% to 5%, from 2% to 6%, from 2% to 8%, from 3% to 5%, from 3% to 6%, from 3% to 8%, from 3% to 10%, from 5% to 7%, from 5% to 8%, from 5% to 10%, from 6% to 8%, from 6% to 10%, from 7% to 9%, from 7% to 10%, or from 9% to 10%).

In some embodiments, the percentage loading of compound 1 or compound 2 is of from about 0.1% to about 60% (w/w) (e.g., from 0.1% to 1%, from 0.1% to 5%, from 0.1% to 10%, from 0.1% to 15%, from 0.1% to 20%, from 0.1% to 25%, from 0.1% to 30%, from 0.1% to 35%, from 0.1% to 40%, from 0.1% to 45%, from 0.1% to 50%, from 0.1% to 55%, from 0.5% to 1%, from 0.5% to 5%, from 0.5% to 10%, from 0.5% to 15%, from 0.5% to 20%, from 0.5% to 25%, from 0.5% to 30%, from 0.5% to 35%, from 0.5% to 40%, from 0.5% to 45%, from 0.5% to 50%, from 0.5% to 55%, from 0.5% to 60%, from 1% to 5%, from 1% to 10%, from 1% to 15%, from 1% to 20%, from 1% to 25%, from 1% to 30%, from 1% to 35%, from 1% to 40%, from 1% to 45%, from 1% to 50%, from 1% to 55%, from 1% to 60%, from 5% to 10%, from 5% to 15%, from 5% to 20%, from 5% to 25%, from 5% to 30%, from 5% to 35%, from 5% to 40%, from 5% to 45%, from 5% to 50%, from 5% to 55%, from 5% to 60%, from 10% to 15%, from 10% to 20%, from 10% to 25%, from 10% to 30%, from 10% to 35%, from 10% to 40%, from 10% to 45%, from 10% to 50%, from 10% to 55%, from 10% to 60%, from 20% to 25%, from 20% to 30%, from 20% to 35%, from 20% to 40%, from 20% to 45%, from 20% to 50%, from 20% to 55%, from 20% to 60%, from 25% to 30%, from 25% to 35%, from 25% to 40%, from 25% to 45%, from 25% to 50%, from 25% to 55%, from 25% to 60%, from 30% to 35%, from 30% to 40%, from 30% to 45%, from 30% to 50%, from 30% to 55%, from 30% to 60%, from 40% to 45%, from 40% to 50%, from 40% to 55%, from 40% to 60%, from 45% to 50%, from 45% to 55%, and from 45% to 60%).

In some embodiments, the carrier includes a lipophilic carrier (e.g., any described herein) and optionally a surfactant carrier (e.g., any described herein).

In some embodiments, following administration of the pharmaceutical composition to subjects (e.g., fed subjects or fasted subjects), the mean bioavailability is greater than about 20% (e.g., greater than 25%, 30%, 35%, 40%, 45%, 50%, 55%, 60%, 65%, 70%, 75%, 80%, 85%, 90%, 95%, or even 99%) or between about 20% to about 90% (e.g., from 20% to 30%, from 20% to 40%, from 20% to 50%, from 20% to 60%, from 20% to 70%, from 20% to 80%, from 20% to 90%, from 30% to 40%, from 30% to 50%, from 30% to 60%, from 30% to 70%, from 30% to 80%, from 30% to 90%, from 40% to 50%, from 40% to 60%, from 40% to 70%, from 40% to 80%, from 40% to 90%, from 50% to 60%, from 50% to 70%, from 50% to 80%, from 50% to 90%, from 60% to 70%, from 60% to 80%, from 60% to 90%, from 70% to 80%, from 70% to 90%, and from 80% to 90%).

In some embodiments, the ratio of the mean bioavailability for fed subjects to the mean bioavailability for fasted subjects is from about 1.0 to about 2.0 (e.g., from 1.0 to 1.1, from 1.0 to 1.2, from 1.0 to 1.3, from 1.0 to 1.4, from 1.0 to 1.5, from 1.0 to 1.6, from 1.0 to 1.7, from 1.0 to 1.8, from 1.0 to 1.9, from 1.3 to 1.4, from 1.3 to 1.5, from 1.3 to 1.6, from 1.3 to 1.7, from 1.3 to 1.8, from 1.3 to 1.9, from 1.3 to 2.0, from 1.5 to 1.6, from 1.5 to 1.7, from 1.5 to 1.8, from 1.5 to 1.9, from 1.5 to 2.0, from 1.7 to 1.8, from 1.7 to 1.9, from 1.7 to 2.0, from 1.8 to 1.9, and from 1.8 to 2.0).

In some embodiments, administration of the pharmaceutical composition to fed and fasted subjects produces a coefficient of variation in Cₘₐₓ and/or AUC_{∞} of less than about 60% (e.g., less than 55%, 50%, 45%, 40%, 35%, 30%, 25%, 20%, and 15%). In particular embodiments, the coefficient of variation in Cₘₐₓ and/or AUC_{∞} is of from about 20% to about 60% (e.g., from 20% to 30%, from 20% to 35%, from 20% to 40%, from 20% to 45%, from 20% to 50%, from 20% to 55%, from 30% to 35%, from 30% to 40%, from 30% to 45%, from 30% to 50%, from 30% to 55%, from 30% to 60%, from 35% to 40%, from 35% to 45%, from 35% to 50%, from 35% to 55%, from 35% to 60%, from 40% to 45%, from 40% to 50%, from 40% to 55%, from 40% to 60%, from 45% to 50%, from 45% to 55%, from 45% to 60%, from 50% to 55%, from 50% to 60%, and from 55% to 60%).

In some embodiments, administration of the pharmaceutical composition to fasted or fed subjects produces a coefficient of variation in Cₘₐₓ and/or AUC_{∞} of less than about 65% (e.g., less than 60%, 55%, 50%, 45%, 40%, 35%, 30%, 25%, 20%, and 15%). In some embodiments, administration of the pharmaceutical composition to fasted or fed subjects produces a coefficient of variation in Cₘₐₓ and/or AUC_{∞} of from about 30% to about 65% (e.g., from 30% to 35%, from 30% to 40%, from 30% to 45%, from 30% to 50%, from 30% to 55%, from 30% to 60%, from 30% to 65%, from 35% to 40%, from 35% to 45%, from 35% to 50%, from 35% to 55%, from 35% to 60%, from 35% to 65%, from 40% to 45%, from 40% to 50%, from 40% to 55%, from 40% to 60%, from 45% to 50%, from 45% to 55%, from 45% to 60%, from 45% to 65%, from 50% to 55%, from 50% to 60%, from 50% to 65%, from 55% to 60%, from 55% to 65%, and from 60% to 65%).

In some embodiments, administration of the pharmaceutical composition to a fasted subject produces a Cₘₐₓ that is greater than about 400 ng/mL (e.g., greater than about 450, 500, 550, 600, 650, 700, 750, or 800 ng/mL and/or up to about 900, 1,000, or 1,500 ng/mL, e.g., from 400 ng/mL to 1,500 ng/mL, from 400 ng/mL to 1,000 ng/mL, from 400 ng/mL to 800 ng/mL, from 400 ng/mL to 700 ng/mL, from 500 ng/mL to 1,500 ng/mL, from 500 ng/mL to 1,000 ng/mL, from 500 ng/mL to 800 ng/mL, and from 500 ng/mL to 700 ng/mL) and/or an AUC_{∞} that is greater than about 4,000 hr*ng/mL (e.g., greater than 4,500, 5,000, 5,500, 6,000, 6,500, 7,000, 7,500, or 8,000 hr*ng/mL and/or up to 8,000 hr*ng/mL, e.g., from 4,000 hr*ng/mL to 8,000 hr*ng/mL, from 4,500 hr*ng/mL to 8,000 hr*ng/mL, from 5,000 hr*ng/mL to 8,000 hr*ng/mL, from 4,000 hr*ng/mL to 7,000 hr*ng/mL, from 4,500 hr*ng/mL to 7,000 hr*ng/mL, and from 5,000 hr*ng/mL to 7,000 hr*ng/mL) for a 225 mg dose of compound 1 or compound 2.

In any of the above aspects, the carrier includes one or more of a lipophilic carrier (e.g., a glyceryl ester of one or more fatty acids, a propylene glycol ester, an ethylene glycol ester, a polyglyceryl ester, and a polyethyloxylated glyceryl ester (e.g., a PEG oleyl glyceryl ester or a PEG linoleyl glycerul ester)), a surfactant carrier (e.g., a polyethoxylated ester of one or more fatty acids, a polyethoxylated alkyl ether, a polyethoxylated glyceryl ester, a polyoxyethylene glyceryl ester of one or more fatty acids, a sorbitan ester, a polyethoxylated sorbitan ester, a polyethoxylated vitamin analog, and an ethoxylated propoxylated block copolymer), or a co-solvent carrier (e.g., ethanol, glycerol, propylene glycol, polyethylene glycol, propylene carbonate, diethylene glycol monoethyl ether, glycofurol, and N-methyl-2-pyrrolidone).

In some embodiments, the lipophilic carrier has a hydrophobic-lipophilic balance of from about 2 to about 10 and the surfactant carrier has a hydrophobic-lipophilic balance of from about 10 to about 20.

In some embodiments, the carrier includes from about 15% to about 50% (w/w) of a lipophilic carrier (e.g., from 15% to 45%, from 15% to 35%, from 15% to 30%, from 15% to 25%, from 20% to 50%, from 20% to 45%, from 20% to 35%, from 20% to 30%, from 20% to 25%, from 30% to 50%, from 30% to 45%, from 30% to 35%, from 40% to 50%, and from 40% to 45%) and of from about 40% to about 80% (w/w) of a surfactant carrier (e.g., from 40% to 75%, from 40% to 70%, from 40% to 65%, from 40% to 60%, from 50% to 80%, from 50% to 75%, from 50% to 70%, from 50% to 65%, from 50% to 60%, from 60% to 80%, from 60% to 75%, from 60% to 70%, from 60% to 65%, from 70% to 80%, and from 70% to 75%).

In some embodiments, the lipophilic carrier is a glyceryl ester of one or more fatty acids having the formula

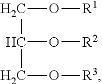

where R¹, R², and R³ are, independently, H or C(O)—X; each X is, independently, optionally substituted C₄₋₂₄ alkyl or optionally substituted C₄₋₂₄ alkenyl; and at least one of R¹, R², or R³ is C(O)—X. In some embodiments, each X is, independently, optionally substituted C₁₂₋₂₄ alkyl or optionally substituted C₁₂₋₂₄ alkenyl. In particular embodiments, C(O)—X is C(O)—(CH₂)ₘ—CH₃; where m is, independently, an integer of from 2 to 12 (e.g., from 2 to 10, from 4 to 10, from 4 to 12, from 6 to 8, from 6 to 10, from 6 to 12, from 8 to 10, from 8 to 12, and from 10 to 12); and at least one of R′, R², or R³ is C(O)—(CH₂)ₘ—CH₃. Exemplary lipophilic carriers include but are not limited to glyceryl butyrates (C_{4:0}), glyceryl caproates (C_{6:0}), glyceryl monocaprylate (C_{8:0}), glyceryl dicaprylate (C_{8:0}), glyceryl tricaprylate (C_{8:0}), glyceryl caprylate (C_{8:0})/caprate (C_{10:0}), glyceryl monocaprate (C_{10:0}), glyceryl dicaprate (C_{10:0}), glyceryl tricaprate (C_{10:0}), glyceryl monolaurate (C_{12:0}), glyceryl dilaurate (C_{12:0}), glyceryl trilaurate (C_{12:0}), glyceryl monomyristate (C_{14:0}), glyceryl dimyristate (C_{14:0}), glyceryl trimyristate (C_{14:0}), glyceryl palmitates (C_{16:0}), glyceryl stearates (C_{18:0}), glyceryl monooleate (C_{18:1}), glyceryl dioleate (C_{18:1}), glyceryl trioleate (C_{18:1}), glyceryl monolinoleate (C_{18:2}), glyceryl dilinoleate (C_{18:2}), glyceryl trilinoleate (C_{18:2}), or a mixture thereof.

In some embodiments, the lipophilic carrier is a propylene glycol ester having the formula

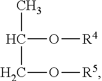

where R⁴ and R⁵ are, independently, H or C(O)—Y; each Y is, independently, optionally substituted C₄₋₂₄ alkyl or optionally substituted C₄₋₂₄ alkenyl; and at least one of R⁴ and R⁵ is C(O)—Y. In some embodiments, each Y is, independently, optionally substituted C₁₂₋₂₄ alkyl or optionally substituted C₁₂₋₂₄ alkenyl. In particular embodiments, C(O)—X is C(O)—(CH₂)ₙ—CH₃; where n is, independently, an integer of from 2 to 12 (e.g., from 2 to 10, from 4 to 10, from 4 to 12, from 6 to 8, from 6 to 10, from 6 to 12, from 8 to 10, from 8 to 12, and from 10 to 12); and at least one of R¹, R², or R³ is C(O)—(CH₂)ₙ—CH₃. Exemplary lipophilic carriers include but are not limited to propylene glycol monocaprylate (C_{8:0}), propylene glycol ester dicaprylate (C_{8:0}), propylene glycol dicaprylocaprate (C_{8:0} and C_{10:0}), propylene glycol monocaprate (C_{10:0}), propylene glycol dicaprate (C_{10:0}), propylene glycol monolaurate (C_{12:0}), propylene glycol dilaurate (C_{12:0}), and a mixture thereof.

In some embodiments, the surfactant carrier is a polyethoxylated ester of one or more fatty acids having the formula R⁶—C(O)O—(CH₂CH₂O)ₚ—R⁷, where R⁶ and R⁷ are, independently, H, optionally substituted C₁₂₋₂₄ alkyl, or optionally substituted C₁₂₋₂₄ alkenyl; p is an integer of from 5 to 50 (e.g., from 5 to 30, from 5 to 35, from 5 to 40, from 5 to 45, from 10 to 30, from 10 to 35, from 10 to 40, from 10 to 45, from 15 to 30, from 15 to 35, from 15 to 40, and from 15 to 45); and at least one of R⁶ or R⁷ is an optionally substituted C₁₂₋₂₄ alkyl or optionally substituted C₁₂₋₂₄ alkenyl (e.g., polyoxyl 40 stearate, polyoxyl 8 stearate, and PEG 15 hydroxystearate).

In some embodiments, the surfactant carrier is a polyethoxylated alkyl ether having the formula R⁸—O—(CH₂CH₂O)_{q}—R⁹, where R⁸ and R⁹ are, independently, H, optionally substituted C₁₂₋₂₄ alkyl, or optionally substituted C₁₂₋₂₄ alkenyl; q is an integer of from 5 to 50 (e.g., from 5 to 30, from 5 to 35, from 5 to 40, from 5 to 45, from 10 to 30, from 10 to 35, from 10 to 40, from 10 to 45, from 15 to 30, from 15 to 35, from 15 to 40, and from 15 to 45); and at least one of R⁸ or R⁹ is an optionally substituted C₁₂₋₂₄ alkyl or optionally substituted C₁₂₋₂₄ alkenyl (e.g., polyoxyl 10 oleoyl ether and PEG 25 cetostearyl ether).

In some embodiments, the carrier includes a mixture of glyceryl monocaprylate and PEG 10 oleoyl ether; a mixture of glyceryl monocaprylate and PEG 15 hydroxystearate; a mixture of glyceryl monocaprylate and polyoxyl 40 stearate; glyceryl monocaprylate; a mixture of propylene glycol monocaprylate and polyoxyl 40 stearate; a mixture of propylene glycol monocaprylate and PEG 10 oleoyl ether; or propylene glycol monocaprylate. In some embodiments, the composition further includes a co-solvent carrier (e.g., ethanol, glycerol, propylene glycol, polyethylene glycol, propylene carbonate, diethylene glycol monoethyl ether, glycofurol, and N-methyl-2-pyrrolidone, e.g., diethylene glycol monoethyl ether and/or N-methyl-2-pyrrolidone).

In some embodiments, the composition further includes from about 0.5% to about 5% (w/w) of a crystallization inhibiting carrier. In particular embodiments, the crystallization inhibiting carrier is selected from the group of a cellulose derivative, a polyvinyl pyrrolidone, a polyvinyl acetate, or a copolymer thereof (e.g., a cellulose acetate phthalate (CAP), methylcellulose acetate phthalate, hydroxypropyl cellulose (HPC), hydroxypropylmethyl cellulose (HPMC), hydroxypropylmethyl cellulose acetate, hydroxypropylmethyl cellulose acetate succinate (HPMCAS), a polyvinyl pyrrolidone (PVP), a polyvinyl acetate (PVA), and a copolymer of a polyvinyl pyrrolidone and a polyvinyl acetate (PVP-PA), or any polymer described herein).

In some embodiments, the carrier, together with the compound 1, compound 2, or the salt thereof, forms a stable emulsion when combined with water to form a solution that is greater than about 50% (w/w) water (e.g., greater than 60%, 70%, 75%, 80%, 85%, or 90% (w/w) water, or from 50% to 90%, from 50% to 85%, from 50% to 80%, from 50% to 75%, from 50% to 70%, from 50% to 60%, from 60% to 90%, from 60% to 85%, from 60% to 80%, from 60% to 75%, from 60% to 70%, from 70% to 90%, from 70% to 85%, from 70% to 80%, from 70% to 75%, from 75% to 90%, from 75% to 85%, from 75% to 80%, from 80% to 90%, from 80% to 85%, and from 85% to 90% (w/w)).

In a third aspect, the invention features a method for reducing the food effect exhibited by compound 1, compound 2, or a salt thereof, following administration to a subject, the method including administering a unit dosage form including any pharmaceutical composition described herein to the subject.

In a fourth aspect, the invention features a method to treat a disease or condition (e.g., pain, epilepsy, or any described herein), the method including administering to a subject (e.g., a fasted subject or a fed subject) in need of such treatment an effective amount of any pharmaceutical composition described herein.

In a fifth aspect, the invention features a method to treat a disease or condition (e.g., pain, epilepsy, or any described herein) modulated by ion channel activity, the method including administering to a subject (e.g., a fasted subject or a fed subject) in need of such treatment an effective amount of any pharmaceutical composition described herein.

In a sixth aspect, the invention features a method of inhibiting an ion channel, the method including contacting a cell (e.g., a cell from a fasted subject or a cell from a fed subject) with any pharmaceutical composition described herein (e.g., an effective amount of any pharmaceutical composition described herein).

In some embodiments of the above aspects, the ion channel is a calcium channel or a sodium channel. In some embodiments, the calcium channel is an N-type calcium channel (e.g., the Ca_{V} 2.2 channel). In some embodiments, the sodium channel is a voltage-gated sodium channel (e.g., the Na_{V}1.7 channel or the Na_{V}1.8, channel).

In some embodiments, the condition is pain, epilepsy, Parkinson's disease, a mood disorder (e.g., a major depressive disorder (e.g., atypical depression, melancholic depression, psychotic major depression, catatonic depression, postpartum depression, seasonal affective disorder, dysthymia, and depressive disorder not otherwise specified (DD-NOS)), recurrent brief depression, minor depressive disorder, or a bipolar disorder), psychosis (e.g., schizophrenia), tinnitus, amyotrophic lateral sclerosis, glaucoma, ischaemia, a spasticity disorder, obsessive compulsive disorder, restless leg syndrome, or Tourette syndrome. In particular embodiments, the condition is pain or epilepsy.

In some embodiments, the pain is inflammatory pain (e.g., caused by rheumatoid arthritis, juvenile idiopathic arthritis, ankylosing spondylitis, psoriatic arthritis, inflammatory bowel disease, primary dysmenorrhea, or endometriosis) or neuropathic pain. In other embodiments, the pain is chronic pain. In further embodiments, the chronic pain is peripheral neuropathic pain (e.g., post-herpetic neuralgia, diabetic neuropathic pain, neuropathic cancer pain, HIV-associated neuropathy, erythromelalgia, failed back-surgery syndrome, trigeminal neuralgia, or phantom limb pain), central neuropathic pain (e.g., multiple sclerosis related pain, Parkinson disease related pain, post-stroke pain, post-traumatic spinal cord injury pain, lumbosacral radiculopathy, cervical radiculopathy, brachial radiculopathy, or pain in dementia), musculoskeletal pain (e.g., osteoarthritic pain or fibromyalgia syndrome), headache (e.g., migraine, cluster headache, tension headache syndrome, facial pain, or headache caused by other diseases), visceral pain (e.g., interstitial cystitis, irritable bowel syndrome, or chronic pelvic pain syndrome), or mixed pain (e.g., lower back pain, neck and shoulder pain, burning mouth syndrome, or complex regional pain syndrome). In still other embodiments, the pain is acute pain. In further embodiments, the acute pain is nociceptive pain or post-operative pain.

In a seventh aspect, the invention features a method of preparing any self-emulsifying pharmaceutical composition described herein, the method including: preparing a solution including compound 1, compound 2, or a salt thereof, and one or more of a lipophilic carrier or a surfactant carrier; heating the solution to the solubilization temperature (e.g., from about 40° C. to about 80° C., such as 40° C. to 50° C., 40° C. to 55° C., 40° C. to 60° C., 40° C. to 65° C., 40° C. to 70° C., 40° C. to 75° C., 50° C. to 55° C., 50° C. to 60° C., 50° C. to 65° C., 50° C. to 70° C., 50° C. to 75° C., 50° C. to 80° C., 60° C. to 65° C., 60° C. to 70° C., 60° C. to 75° C., 60° C. to 80° C., 70° C. to 75° C., and 70° C. to 80° C.) for the lipophilic carrier or the surfactant carrier; and mixing the solution to form the self-emulsifying pharmaceutical composition (e.g., where the self-emulsifying pharmaceutical composition includes a self-emulsifying carrier together with the compound 1, compound 2, or the salt thereof). In some embodiments, the method further includes: cooling the solution; and filling the unit dosage form with the solution. In some embodiments, the solution further includes a crystallization inhibiting carrier (e.g., any described herein). In some embodiments, the method further includes immediately (e.g., within one hour, two hours, three hours, five hours, or six hours after preparing the composition) administering the self-emulsifying pharmaceutical composition to the subject.

In some embodiments, the solution includes a lipophilic carrier and the lipophilic carrier is a glyceryl ester of one or more fatty acids having the formula

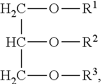

where R¹, R², and R³ are as described herein.

In other embodiments, the solution includes a lipophilic carrier and the lipophilic carrier is a propylene glycol ester having the formula

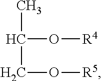

where R⁴ and R⁵ are as described herein.

In some embodiments, the solution includes a surfactant carrier and the surfactant carrier is a polyethoxylated ester of one or more fatty acids having the formula R⁶—C(O)O—(CH₂CH₂O)ₚ—R⁷, where R⁶ and R⁷ are as described herein.

In some embodiments, the solution includes a surfactant carrier and the surfactant carrier is a polyethoxylated alkyl ether having the formula R⁸—O—(CH₂CH₂O)_{q}—R⁹, where R⁸ and R⁹ are as described herein.

In some embodiments, the solution includes a mixture of glyceryl monocaprylate and PEG 10 oleoyl ether; a mixture of glyceryl monocaprylate and PEG 15 hydroxystearate; a mixture of glyceryl monocaprylate and polyoxyl 40 stearate; glyceryl monocaprylate; a mixture of propylene glycol monocaprylate and polyoxyl 40 stearate; a mixture of propylene glycol monocaprylate and PEG 10 oleoyl ether; or propylene glycol monocaprylate.

In some embodiments, the solution further includes a co-solvent carrier (e.g., ethanol, glycerol, propylene glycol, polyethylene glycol, propylene carbonate, diethylene glycol monoethyl ether, glycofurol, and N-methyl-2-pyrrolidone).

In any of the aspects described herein, the composition includes compound 1 or a salt thereof.

In any of the aspects described herein, the composition includes compound 2 or a salt thereof.

In any of the above aspects, the unit dosage form includes from about 20 mg to about 100 mg (e.g., about 75 mg) of compound 1, compound 2, or a salt thereof.

In any of the above aspects, the compound 1, compound 2, or the salt thereof, is the hydrochloride salt of compound 1, the hydrochloride salt of compound 2, the free base form of compound 1, or the free base form of compound 2.

In any of the above aspects, the compound 1 is the free base form of compound 1.

In any of the above aspects, the unit dosage form is a hard gelatin capsule, a hard hydroxypropyl methylcellulose capsule, or a soft gelatin capsule.

In any of the methods described herein, the self-emulsifying pharmaceutical composition is prepared and then immediately (e.g., within one hour, two hours, three hours, five hours, or six hours) administered to the subject. In any of the methods described herein, the self-emulsifying pharmaceutical composition is prepared, stored under refrigerated conditions (e.g., from about 36° F. to about 46° F. (about 2° C. to about 8° C.)), and then administered (e.g., within one hour, two hours, three hours, five hours, six hours, one day, one week, two weeks, three weeks, one month, two months, or three months) to the subject.

In any of the above aspects, the unit dosage form includes from about 20 mg to about 250 mg of compound 1, compound 2, or a salt thereof, such as from 20 mg to 30 mg, from 20 mg to 40 mg, from 20 mg to 50 mg, from 20 mg to 75 mg, from 20 mg to 100 mg, from 20 mg to 125 mg, from 20 mg to 150 mg, from 20 mg to 175 mg, from 20 mg to 200 mg, from 20 mg to 225 mg, from 30 mg to 40 mg, from 30 mg to 50 mg, from 30 mg to 75 mg, from 30 mg to 100 mg, from 30 mg to 125 mg, from 30 mg to 150 mg, from 30 mg to 175 mg, from 30 mg to 200 mg, from 30 mg to 225 mg, from 30 mg to 250 mg, from 40 mg to 50 mg, from 40 mg to 75 mg, from 40 mg to 100 mg, from 40 mg to 125 mg, from 40 mg to 150 mg, from 40 mg to 175 mg, from 40 mg to 200 mg, from 40 mg to 225 mg, from 40 mg to 250 mg, from 50 mg to 75 mg, from 50 mg to 100 mg, from 50 mg to 125 mg, from 50 mg to 150 mg, from 50 mg to 175 mg, from 50 mg to 200 mg, from 50 mg to 225 mg, from 50 mg to 250 mg, from 60 mg to 75 mg, from 60 mg to 100 mg, from 60 mg to 125 mg, from 60 mg to 150 mg, from 60 mg to 175 mg, from 60 mg to 200 mg, from 60 mg to 225 mg, from 60 mg to 250 mg, from 70 mg to 75 mg, from 70 mg to 100 mg, from 70 mg to 125 mg, from 70 mg to 150 mg, from 70 mg to 175 mg, from 70 mg to 200 mg, from 70 mg to 225 mg, from 70 mg to 250 mg, from 80 mg to 100 mg, from 80 mg to 125 mg, from 80 mg to 150 mg, from 80 mg to 175 mg, from 80 mg to 200 mg, from 80 mg to 225 mg, from 80 mg to 250 mg, from 90 mg to 100 mg, from 90 mg to 125 mg, from 90 mg to 150 mg, from 90 mg to 175 mg, from 90 mg to 200 mg, from 90 mg to 225 mg, from 90 mg to 250 mg, from 100 mg to 125 mg, from 100 mg to 150 mg, from 100 mg to 175 mg, from 100 mg to 200 mg, from 100 mg to 225 mg, and from 100 mg to 250 mg.

In any of the above aspects, the unit dosage form is administered to achieve a daily amount of from about 25 mg to about 1,600 mg (e.g., from 40 mg to 1,600 mg, from 40 mg to 1,000 mg, from 40 mg to 800 mg, from 40 mg to 700 mg, from 40 mg to 600 mg, from 40 mg to 500 mg, from 40 mg to 400 mg, from 40 mg to 300 mg, from 40 mg to 200 mg, from 50 mg to 1,600 mg, from 50 mg to 1,000 mg, from 50 mg to 800 mg, from 50 mg to 700 mg, from 50 mg to 600 mg, from 50 mg to 500 mg, from 50 mg to 400 mg, from 50 mg to 300 mg, from 50 mg to 200 mg, from 60 mg to 1,600 mg, from 60 mg to 1,000 mg, from 60 mg to 800 mg, from 60 mg to 700 mg, from 60 mg to 600 mg, from 60 mg to 500 mg, from 60 mg to 400 mg, from 60 mg to 300 mg, from 60 mg to 200 mg, from 80 mg to 1,600 mg, from 80 mg to 1,000 mg, from 80 mg to 800 mg, from 80 mg to 700 mg, from 80 mg to 600 mg, from 80 mg to 500 mg, from 80 mg to 400 mg, from 80 mg to 300 mg, from 80 mg to 200 mg, from 100 mg to 1,600 mg, from 100 mg to 1,000 mg, from 100 mg to 800 mg, from 100 mg to 700 mg, from 100 mg to 600 mg, from 100 mg to 500 mg, from 100 mg to 400 mg, from 100 mg to 300 mg, from 100 mg to 200 mg, from 150 mg to 1,600 mg, from 150 mg to 1,000 mg, from 150 mg to 800 mg, from 150 mg to 700 mg, from 150 mg to 600 mg, from 150 mg to 500 mg, from 150 mg to 400 mg, from 150 mg to 300 mg, and from 150 mg to 200 mg, such as from 40 mg to 800 mg and from 80 mg to 320 mg) of compound 1, compound 2, or a salt thereof. In additional aspects, the unit dosage form is administered to achieve a daily amount of up to 1,600 mg (e.g., up to 1,500 mg, up to 1,250 mg, up to 1,000 mg, up to 750 mg, up to 500 mg, up to 450 mg, up to 400 mg, up to 350 mg, up to 300 mg, up to 250 mg, up to 200 mg, up to 150 mg, up to 100 mg, and up to 50 mg, preferably up to 400 mg) or a daily amount of from about 50 mg to about 1,600 mg (e.g., from 150 mg to 200 mg, from 150 mg to 225 mg, from 150 mg to 500 mg, from 150 mg to 750 mg, from 150 mg to 900 mg, from 150 mg to 1,000 mg, from 150 mg to 1,250 mg, from 150 mg to 1,500 mg, from 150 mg to 1,600 mg, from 200 mg to 225 mg, from 200 mg to 500 mg, from 200 mg to 750 mg, from 200 mg to 900 mg, from 200 mg to 1,000 mg, from 200 mg to 1,250 mg, from 200 mg to 1,500 mg, from 200 mg to 1,600 mg, from 225 mg to 500 mg, from 225 mg to 750 mg, from 225 mg to 900 mg, from 225 mg to 1,000 mg, from 225 mg to 1,250 mg, from 225 mg to 1,500 mg, from 225 mg to 1,600 mg, from 500 mg to 750 mg, from 500 mg to 900 mg, from 500 mg to 1,000 mg, from 500 mg to 1,250 mg, from 500 mg to 1,500 mg, from 500 mg to 1,600 mg, from 750 mg to 900 mg, from 750 mg to 1,000 mg, from 750 mg to 1,250 mg, from 750 mg to 1,500 mg, from 750 mg to 1,600 mg, from 900 mg to 1,000 mg, from 900 mg to 1,250 mg, from 900 mg to 1,500 mg, from 900 mg to 1,600 mg, from 1,000 mg to 1,250 mg, from 1,000 mg to 1,500 mg, from 1,000 mg to 1,600 mg, from 1,250 mg to 1,500 mg, from 1,250 mg to 1,600 mg, and from 1,500 mg to 1,600 mg, e.g., about 225 mg) of compound 1, compound 2, or a salt thereof. In further aspects, the unit dosage form is administered one to five times daily (e.g., one, two, three, four, or five times daily).

### DEFINITIONS

As used herein, “about” means +/−10% of the recited value.

As used herein, “bioavailability” refers to the fraction of drug absorbed following administration to a subject or patient under a fasted state. Under fasted states, the bioavailability of compound 1, compound 2, or a salt thereof, formulated as described herein is at least about 15%, but may be greater than 20%, 25%, 30%, 35%, 40%, 45%, or 50% of the dose administered.

By “coefficient of variation” is meant the arithmetic standard deviation divided by the arithmetic mean for a particular pharmacokinetic parameter, where the data is obtained from a pharmacokinetic study involving 10, 12, or more subjects or patients.

By “mean” is meant the arithmetic mean for a particular pharmacokinetic parameter, where the data is obtained from a pharmacokinetic study involving 10, 12, or more subjects or patients.

By “Cₘₐₓ” is meant the mean peak concentration of a drug achieved in plasma after dosing.

By “Tₘₐₓ” is meant the mean time after oral administration of a drug when the maximum plasma concentration of the drug or Cₘₐₓ is reached.

By “AUC_{∞},” “AUC_{0-∞},” or “Area Under the Curve_{∞}” is meant the mean integrated area under the curve for the plasma concentration of a drug, versus time from t=0 to ∞ following dosing.

By “food effect” is meant is meant a difference between any one or more of Cₘₐₓ, Tₘₐₓ, AUC_{∞}, and bioavailability for a drug, administered under fasted states in comparison to the drug administered under fed states.

As used herein, “reducing the food effect” refers to narrowing the difference between any one of Cₘₐₓ, Tₘₐₓ, AUC_{∞}, and bioavailability for a drug administered under fasted states in comparison to the drug administered under fed states.

By “fasted” or “fasted states” is meant a subject has not eaten for at least about four hours prior and about four hours subsequent to drug administration.

By “fed” or “fed states” is meant a subject has eaten within about 30 minutes prior to drug administration. The meal can be a fatty meal, and the resulting mean pharmacokinetic parameters can be characteristic of consuming a fatty meal. For example, the “fed state” can be a human who has eaten a United States Food and Drug Administration (FDA) standard high fat breakfast (or another meal containing a comparable quantity of fat and calories) within 30 minutes prior to drug administration. A typical FDA standard breakfast consists of 2 eggs fried in butter, 2 strips of bacon, 2 slices of toast with butter, 4 ounces of hash brown potatoes, and 8 ounces of whole milk. The meal is high in both fat (approximately 50% of total calorie content of the meal) and calories (approximately 800-1,000 calories).

By “emulsion” is meant a suspension having a continuous aqueous phase and a dispersed lipid phase including one or more carriers and a drug. Emulsions may be partially, temporarily and/or completely stable. By “stable emulsion” is meant an emulsion that will not separate into its components under the conditions for which it was made.

As used herein, the term “self-emulsifying” refers to a compound, composition, or formulation that, upon contact with an aqueous medium, spontaneously forms an emulsion. The self-emulsifying compositions and formulations of the invention can form stable emulsions in solutions that are greater than 50%, 60%, 70%, 75%, 80%, 85%, or 90% (w/w) water.

By “pharmaceutically acceptable carrier” is meant a carrier suitable for pharmaceutical formulation. Pharmaceutically acceptable carriers may be self-emulsifying or participate in self-emulsifying compositions or formulations. In this regard, these carriers may be referred to as self-emulsifying carrier systems or be present in a self-emulsifying state.

By “lipophilic carrier” is meant a pharmaceutically acceptable carrier having a hydrophobic-lipophilic balance of from about 2 to about 10 (e.g., from 2 to 9, from 2 to 8, from 2 to 7, from 2 to 6, from 2 to 5, from 2 to 4, from 2 to 3, from 3 to 10, from 3 to 9, from 3 to 8, from 3 to 7, from 3 to 6, from 3 to 5, from 3 to 4, from 4 to 10, from 4 to 9, from 4 to 8, from 4 to 7, from 4 to 6, from 4 to 5, from 5 to 10, from 5 to 9, from 5 to 8, from 5 to 7, from 5 to 6, from 6 to 10, from 6 to 9, from 6 to 8, from 6 to 7, from 7 to 10, from 7 to 9, from 7 to 8, from 8 to 10, from 8 to 9, and from 9 to 10, such as from 4.5 to 7.0, from 4.5 to 6.5, from 4.5 to 6.0, from 4.5 to 5.5, from 5.0 to 6.5, from 5.5 to 7.0, from 5.5 to 6.5, and from 5.5 to 6.0). Useful methods for determining hydrophobic-lipophilic balance (“HLB”) are provided herein.

By “surfactant carrier” is meant a pharmaceutically acceptable carrier having a hydrophobic-lipophilic balance of from about 10 to about 20 (e.g., from 10 to 19, from 10 to 18, from 10 to 17, from 10 to 16, from 10 to 15, from 10 to 14, from 10 to 13, from 10 to 12, from 10 to 11, from 11 to 20, from 11 to 19, from 11 to 18, from 11 to 17, from 11 to 16, from 11 to 15, from 11 to 14, from 11 to 13, from 11 to 12, from 12 to 20, from 12 to 19, from 12 to 18, from 12 to 17, from 12 to 16, from 12 to 15, from 12 to 14, from 12 to 13, from 13 to 20, from 13 to 19, from 13 to 18, from 13 to 17, from 13 to 16, from 13 to 15, from 13 to 14, from 14 to 20, from 14 to 19, from 14 to 18, from 14 to 17, from 14 to 16, from 14 to 15, from 15 to 20, from 15 to 19, from 15 to 18, from 15 to 17, from 15 to 16, from 16 to 20, from 16 to 19, from 16 to 18, from 16 to 17, from 17 to 20, from 17 to 19, from 17 to 18, from 18 to 20, from 18 to 19, and from 19 to 20, such as from 11 to 18, from 11 to 17, from 11 to 16, from 11.5 to 18, from 11.5 to 17, and from 11.5 to 16). Useful methods for determining hydrophobic-lipophilic balance (“HLB”) are provided herein.

The term “unit dosage form” refers to a physically discrete unit suitable as a unitary dosage, such as a tablet, caplet, hard capsule, or soft capsule, each unit containing a predetermined quantity of a drug.

The term “alkylene” and the prefix “alk-,” as used herein, represent a saturated divalent hydrocarbon group derived from a straight or branched chain saturated hydrocarbon by the removal of two hydrogen atoms, and is exemplified by methylene, ethylene, isopropylene, and the like. The term “C_{x-y} alkylene” and the prefix “C_{x-y} alk-” represent alkylene groups having between x and y carbons. Exemplary values for x are 1, 2, 3, 4, 5, and 6, and exemplary values for y are 2, 3, 4, 5, 6, 7, 8, 9, or 10. In some embodiments, the alkylene can be further substituted with 1, 2, 3, or 4 substituent groups as defined herein for an alkyl group.

The term “alkyl,” as used herein, is inclusive of both straight chain and branched chain saturated groups from 1 to 24 carbons (e.g., 4 to 24 carbons or 12 to 24 carbons), unless otherwise specified. Alkyl groups are exemplified by methyl, ethyl, n- and iso-propyl, n-, sec-, iso- and tert-butyl, neopentyl, hexyl, heptyl, octyl, nonyl, decyl, undecyl, dodecyl, icosyl, and the like, and may be optionally substituted with one, two, three, or, in the case of alkyl groups of two carbons or more, four substituents independently selected from the group consisting of: (1) C₁₋₆ alkoxy; (2) C₁₋₆ alkylsulfinyl; (3) amino; (4) C₆₋₁₀ aryl-C₁₋₆ alkoxy, where an aryl group is attached to an alkylene group, which in turn is attached to the parent molecular group through an oxygen atom; (5) azido; (6) halo; (7) (C₂₋₉heterocyclyl)oxy; (8) hydroxy; (9) nitro; (10) oxo (e.g., carboxyaldehyde or acyl); (11) C₁₋₇ spirocyclyl; (12) thioalkoxy; (13) thiol; (14) —CO₂R^{A′}, where R^{A′} is selected from the group consisting of (a) C₁₋₆ alkyl, (b) C₆₋₁₀ aryl, (c) hydrogen, and (d) C₁₋₆ alk-C₆₋₁₀ aryl, which represents an aryl group attached to the parent molecular group through an alkylene group; (15) —C(O)NR^{B′}R^{C′}, where each of R^{B′} and R^{C′} is, independently, selected from the group consisting of (a) hydrogen, (b) C₁₋₆ alkyl, (c) C₆₋₁₀ aryl, and (d) C₁₋₆ alk-C₆₋₁₀ aryl; (16) —SO₂R^{D′}, where R^{D′} is selected from the group consisting of (a) C₁₋₆ alkyl, (b) C₆₋₁₀ aryl, and (c) C₁₋₆ alk-C₆₋₁₀ aryl; and (17) —SO₂NR^{E′}R^{F′}, where each of R^{E′} and R^{F′} is, independently, selected from the group consisting of (a) hydrogen, (b) C₁₋₆ alkyl, (c) C₆₋₁₀ aryl and (d) C₁₋₆ alk-C₆₋₁₀ aryl. In some embodiments, each of these groups can be further substituted by any exemplary alkyl substituent groups described herein. For example, the alkylene group of a C₁-alkaryl can be further substituted with an oxo group to afford the respective aryloyl substituent. Additional exemplary alkyl groups include but are not limited to tetracosylic (C_{24:0}), tricosylic (C_{23:0}), behenic (C_{22:0}), heneicosylic (C_{21:0}), arachidic (C_{20:0}), nonadecylic (C_{19:0}), stearic (C_{18:0}), margaric (C_{17:0}), palmitic (C_{16:0}), pentadecylic (C_{15:0}), myristic (C_{14:0}), tridecylic (C_{13:0}), laurie (C_{12:0}), undecylic (C_{11:0}), capric (C_{10:0}), pelargonic (C_{9:0}), caprylic (C_{8:0}), enanthic (C_{7:0}), and caproic (C_{6:0}).

The term “alkenyl,” as used herein, represents monovalent straight or branched chain hydrocarbon groups of, unless otherwise specified, from 1 to 24 carbons (e.g., 4 to 24 carbons or 12 to 24 carbons) containing one or more carbon-carbon double bonds and is exemplified by ethenyl, 1-propenyl, 2-propenyl, 2-methyl-1-propenyl, 1-butenyl, 2-butenyl, and the like. Alkenyl groups may be optionally substituted with 1, 2, 3, or 4 substituent groups that are selected, independently, from aryl, cycloalkyl, or heterocyclyl, or any of the exemplary alkyl substituent groups described herein. Additional exemplary alkenyl group include but are not limited to docosenyl (C_{22:1}), arachidonic (C_{20:4}), dihomo-γ-linolenic (C_{20:3}), eicosenoic (C_{20:1}), stearidonic (C_{18:4}), γ-linolenic (C_{18:3}), α-linolenic (C_{18:3}), linoleic (C_{18:2}), oleic (C_{18:1}), undecenyl (C_{11:1}), decenyl (C_{10:1}), nonenyl (C_{9:1}), and octenyl (C_{8:1}), where the number after the colon indicates the number of double bonds.

The term “fatty acid,” as used herein, represents a carboxylic acid group (—CO₂H), either with or without the hydrogen, attached to an alkyl group, such as any described herein. Exemplary fatty acids include short chain fatty acids with 1 to 6 carbons; medium chain fatty acids with 6 to 12 carbons; long chain fatty acids with 12 to 22 carbons; and very long chain fatty acids having more than 22 carbons, as well as any described herein. Fatty acids may be optionally substituted with 1, 2, 3, or 4 substituent groups, such as any of the exemplary alkyl substituent groups described herein.

As used herein, the term “administration” or “administering” refers to peroral (e.g., oral) administration of a drug to a subject or patient.

By “effective” amount is meant the amount of a drug sufficient to treat, prevent, or ameliorate a condition in a subject or patient. The effective amount of compound 1, compound 2, or salt thereof, used to practice the present invention for therapeutic management of a condition varies depending upon one or more of the manner of administration, the age, body weight, sex, and/or general health or malady of the patient. The prescribers will primarily decide the appropriate amount and dosage regimen. Such amount is referred to as an “effective” amount.

As used herein, and as well understood in the art, “to treat” a condition or “treatment” of the condition (e.g., the conditions described herein such as pain (e.g., chronic or acute pain), epilepsy, Alzheimer's disease, Parkinson's disease, cardiovascular disease, diabetes, cancer, sleep disorders, obesity, mood disorders, psychosis such as schizophrenia, tinnitus, amyotrophic lateral sclerosis, glaucoma, ischaemia, spasticity disorders, obsessive compulsive disorder, restless leg syndrome, Tourette syndrome, overactive bladder, renal disease, neuroprotection, addiction, or male birth control) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

Other features and advantages of the invention will be apparent from the following detailed description, the drawings, and the claims.

### BRIEF DESCRIPTION OF THE DRAWINGS

FIGS. 1A-1D are phase diagrams for lipophilic carrier glyceryl monocaprylate and various surfactant carriers, including PEG 10 oleoyl ether (FIG. 1A), PEG 8 stearate (FIG. 1B), PEG 15 hydroxystearate (FIG. 1C), and polyoxyl 40 stearate (FIG. 1D). Microemulsion regions are shown in dark gray.

FIGS. 2A-2B are phase diagrams for lipophilic carrier propylene glycol monocaprylate and various surfactant carriers, including PEG 10 oleoyl ether (FIG. 2A) and polyoxyl 40 stearate (FIG. 2B). Microemulsion regions are shown in dark gray.

FIGS. 3A-3B are graphs for dissolution of formulation B (FIG. 3A) and formulation E2 (FIG. 3B) at pH 5 and 6.5.

FIGS. 4A-4C are graphs showing mean concentration of compound 1 in plasma for various formulations in an in vivo studies in rats. Data are shown for formulation B (FIG. 4A), formulation E2 (FIG. 4B), and control (FIG. 4C) with doses of 10 mg/kg, 30 mg/kg, and 100 mg/kg.

FIGS. 5A-5C are graphs showing mean concentration of compound 1 in plasma for various doses in an in vivo study in rats. Data are shown for formulations B, E2, and control with doses of 10 mg/kg (FIG. 5A), 30 mg/kg (FIG. 5B), and 100 mg/kg (FIG. 5C).

FIG. 6 is a graph showing mean concentration of compound 1 in plasma for various formulations in an in vivo study in dogs with a 10 mg/kg dose. Data are shown for formulations B, E2, and control.

### DETAILED DESCRIPTION

The invention provides methods for treating conditions related to N-type calcium channels, involving administration of 1-(4-benzhydrylpiperazin-1-yl)-3,3-diphenylpropan-1-one (compound 1), a metabolite thereof (e.g., 1-(3,3-diphenylpropanoyl)piperazine (compound 2), or a salt thereof. Non-limiting examples of conditions treatable by this administration are pain and epilepsy.

Compound 1 is a potent and selective N-type calcium channel antagonist, and doses of up to 1,600 mg provided no adverse effects. Yet, compound 1 is also known to have decreased oral bioavailability in the fasted state, as compared to the fed state. Accordingly, the invention provides formulations to increase the oral bioavailability or to reduce patient-to-patient variability in pharmacokinetic behavior of compound 1, compound 2, or a salt thereof. These formulations include use of a self-emulsifying carrier system to provide stable emulsions capable of dissolving therapeutically effective amounts of compound 1, compound 2, or a salt thereof.

### Previously Determined Characteristics of Compound 1

Compound 1 has the following previously determined characteristics:

(i) physical appearance: white to off-white powder;

(ii) solubility: slightly soluble in water (0.2 μg/ml to 2.0 μg/ml at pH of 6.8, 0.03 μg/ml at pH of 6.5, 1.5 μg/ml at pH of 6.5 in FaSSIF, and 55 μg/ml at pH of 1.0) and soluble in propylene glycol, ethanol, and tetrahydrofuran;

(iii) pKa: 5.4;

(iv) log P: 2.6;

(v) M.P.: 123° C. (for free base) and 126° C. (for HCl salt);

(vi) hygroscopicity (for free base): 0.09% at 70% relative humidity (RH);

(vii) potential isomerism: none;

(viii) Tₘₐₓ for HCl salt: <2 hours (fasted state), 2-5 hours (fed state with normal fat meal), and 4-5 hours (fed state with high fat meal); and

(ix) t_{1/2} for absorption for HCl salt: 0.05-0.17 hours (fasted state), 0.5-1.6 hours (fed state with normal fat meal), and 0.8-2.8 hours (fed state with high fat meal). The fed-fasted pharmacokinetic data were collected using a “normal fat” meal consisting of toast with 1 pat of butter, a banana, 2% milk, apple juice, and Honey Nut Cheerios®, where the meal has about 501 calories with 99 calories from fat, 346 calories from carbohydrates, and 56 calories from protein; and using a “high fat” meal consisting of 2 eggs fried in butter, 2 strips of bacon, 2 slices of toast with butter, 4 ounces of hash brown potatoes and 8 ounces of whole milk, where the meal has about 1,000 calories with 500-600 calories from fat, 250 calories from carbohydrates, and 150 calories, from protein.

The relative oral bioavailability of compound 1 (HCl salt) has been previously determined in various fed and fasted states in a micronized formulation. This micronized formulation included the following for a 100 mg dose in a #1 HPMC (white opaque) capsule: compound 1 HCl micronized (100.00 mg), Lactose Fast Flo® Fast Flo® composed of a spray-dried mixture of crystalline and amorphous lactose monohydrate (184.80 mg); sodium starch glycolate (19.20 mg); polysorbate 80 (14.40 mg); purified water (used as the granulating fluid and was removed during processing) (QS); and magnesium stearate (1.60 mg), where the resultant fill weight was 320.00 mg. For a 25 mg dose in a #1 gelatin (white opaque) capsule, the formulation included the following: compound 1 HCl micronized (25.00 mg), Lactose Fast Flo® Fast Flo® composed of a spray-dried mixture of crystalline and amorphous lactose monohydrate (264.60 mg); sodium starch glycolate (19.20 mg); polysorbate 80 (9.60 mg); purified water (used as the granulating fluid and was removed during processing) (QS); and magnesium stearate (1.60 mg), where the resultant fill weight was 320.00 mg.

For the micronized formulation, typical values for relative bioavailability included 0.682 for a fasted state relative to a fed state with a normal fat meal; 1.48 for a 25 mg capsule relative to a 100 mg capsule; 5.60 for a fed state with a high fat meal relative to a fed state with a normal fat meal; and

$$1 - 0.363 \times {log}_{10} ( \frac{dose}{100} )$$

as a function of dose (mg) relative to a 100 mg dose. Typical values were computed as 100% ×√ω², where ω²=variance (eta), and 68% of the study population were within the range of these typical values. Relative bioavailability values derived from the provided typical values are provided in Table 1.

TABLE 1

| | | Meal* | | |
|---|---|---|---|---|
| Dosage form | Dose (mg) | Fasted | Normal fat | High fat |
| 100 mg | 100 | 0.682 | 1.000 | 5.600 |
| capsule | 200 | 0.607 | 0.891 | 4.988 |
| | 400 | 0.533 | 0.781 | 4.376 |
| | 800 | 0.458 | 0.672 | 3.764 |
| | 1,600 | 0.384 | 0.563 | 3.152 |
| 25 mg | 25 | 1.230 | NA | 10.10 |
| capsule | 50 | 1.120 | NA | 9.19 |
| | 100 | 1.009 | NA | 8.29 |
| | 200 | 0.899 | NA | 7.38 |
| | 400 | 0.789 | NA | 6.48 |
| | 800 | 0.678 | NA | 5.57 |
| | 1,600 | 0.568 | NA | 4.67 |

*Values are expressed relative to the 100 mg dose administered with a normal fat meal.

### Synthesis of Compound 1

Compound 1, or a salt thereof, can be synthesized by any useful method, including those described in U.S. Pat. Nos. 6,294,533; 6,387,897; 6,492,375; 6,617,322; 6,949,554; 6,951,862; and 7,064,128; and U.S. Patent Publication Nos. 2006/0084660 and 2004/0259866, incorporated herein by reference in their entirety.

Scheme 1 provides an exemplary schematic for the synthesis of compound 1 (free base). Briefly, the first and second steps provide purified 3,3-diphenylpropionic acid, and these steps can optionally include a recrystallization step in ethyl acetate/heptanes (70/30). Then, the third step provides compound 1, and this step can optionally include use of a toluene azeotrope to remove residual solvents, such as ethanol, tetrahydrofuran (THF), ethyl acetate, commercial grade heptanes, toluene, or isopropanol.

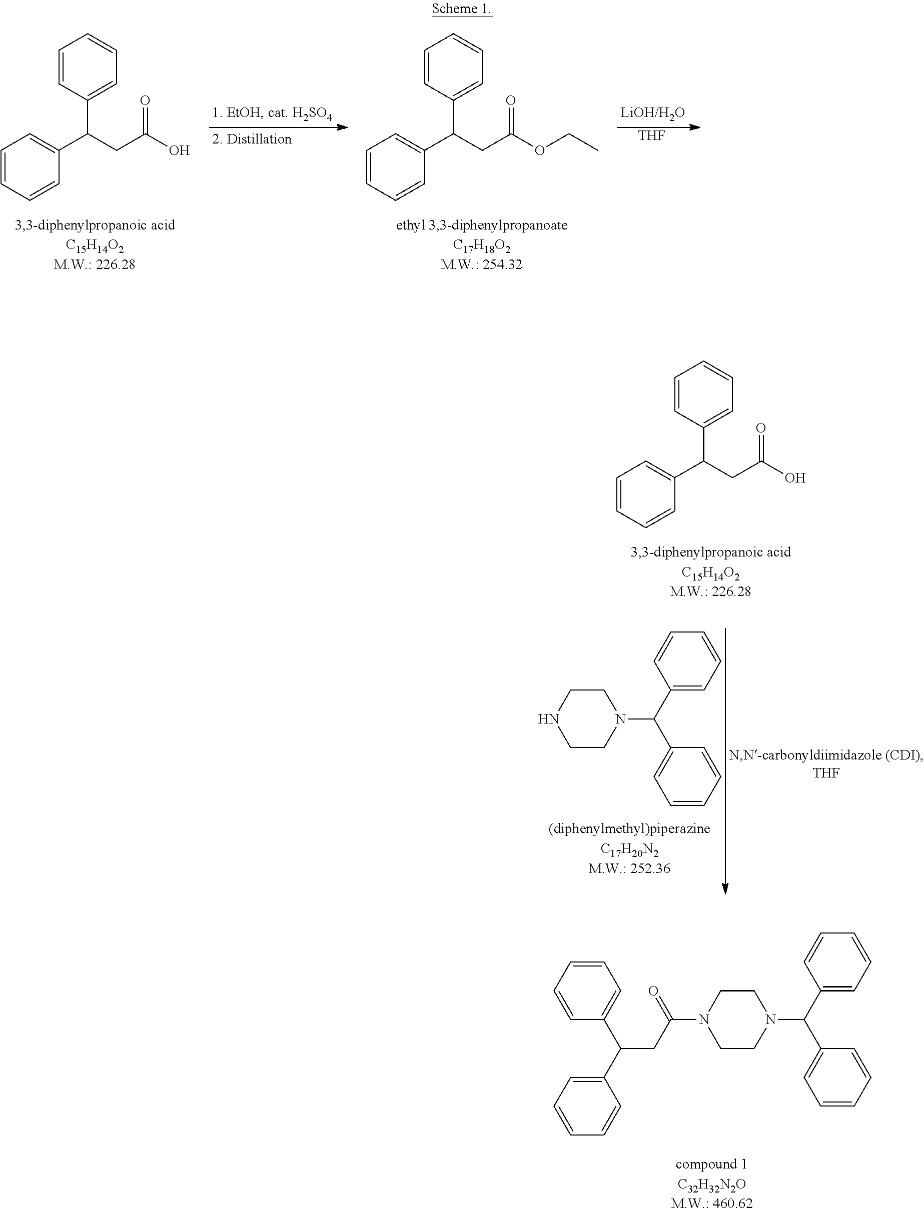

Synthesis of compound 2

Compound 2, or a salt thereof, can be synthesized by any useful method, including those described in U.S. Pat. Nos. 6,011,035; 6,951,862; and 7,186,726; U.S. Patent Application Publication Nos. 2006/0084660 and 2004/0259866; International Publications Nos. WO 2008/066803, WO 2011/006073, and WO 2007/118323; J. Am. Chem. Soc. 77:3142, 1955; and J. Am. Pharm. Assoc. 46:279, 1957; incorporated herein by reference in their entirety.

Scheme 2 provides an exemplary schematic for the synthesis of compound 2 (free base). Briefly, the first step provides a protected carbamate compound. The second step provides compound 2. These steps can optionally include a purification step (e.g., where the starting material, 3,3-diphenylpropanoic acid, can be optionally purified, as shown in Scheme 1), a recrystallization step in ethyl acetate/heptanes (e.g., in a ratio of 70/30), or use of a toluene azeotrope to remove residual solvents, such as ethanol, tetrahydrofuran (THF), ethyl acetate, commercial grade heptanes, toluene, or isopropanol.

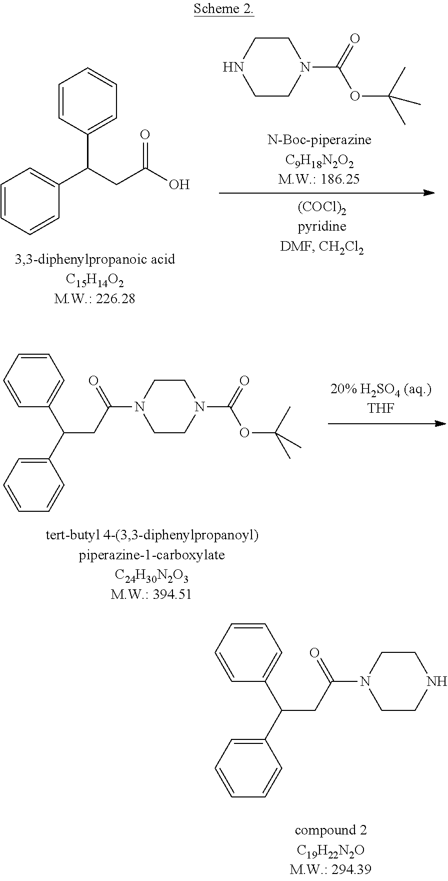 [SMILES: O=C(O)CC(c1ccccc1)c1ccccc1 ; CC(C)(C)OC(=O)N1CCNCC1 ; CC(C)(C)OC(=O)N1CCN(C(=O)CC(c2ccccc2)c2ccccc2)CC1 ; O=C(CC(c1ccccc1)c1ccccc1)N1CCNCC1]

### Self-Emulsifying Carriers

Self-emulsifying carriers are those carriers, whether alone or in combination with compound 1 or compound 2, that are capable of forming oil-in-water emulsions or microemulsions. Two or more self-emulsifying carriers can be used to form higher order formulations, such as binary, ternary, or quaternary compositions or formulations. Generally, the compositions or formulations include a homogenous mixture of one or more carriers of different types. Exemplary types of carriers include lipophilic carriers, surfactant carriers, and co-solvent carriers.

Lipophilic Carriers

The formulations of the invention can include one or more lipophilic carriers. Lipophilic carriers generally include long-chain and medium-chain fatty acid mono-, di- and triglycerides having various degrees of saturation, natural oils, and hydrolyzed oils (e.g., hydrolyzed vegetable oil). These carriers generally have relatively low hydrophobic-lipophilic balance (HLB) (e.g., 2 to 10).

In some examples, the lipophilic carrier can be a glyceryl ester of a fatty acid having the formula

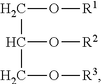

where R¹, R², and R³ are, independently, H or C(O)—X; each X is, independently, optionally substituted C₄₋₂₄ alkyl or optionally substituted C₄₋₂₄ alkenyl (e.g., C₁₂₋₂₄ alkyl or C₁₂₋₂₄ alkenyl); and at least one of R′, R², or R³ is C(O)—X. Glyceryl monoesters have one C(O)—X group, where glyceryl diesters and glyceryl triesters have two or three such groups, respectively.

In some embodiments, C(O)—X is C(O)—(CH₂)ₘ—CH₃, where m is, independently, an integer of from 2 to 12. Exemplary glyceryl esters of a fatty acid include glyceryl mono-, di-, and triesters, where m is 2 (glyceryl butyrates (C_{4:0})), 4 (glyceryl caproates (C_{6:0}), 6 (glyceryl caprylates (C_{8:0})), 8 (glyceryl caprates (C_{10:0}), 10 (glyceryl laurates (C_{12:0})), 12 (glyceryl myristates (C_{14:0})), 14 (glyceryl palmitates (C_{16:0})), and 16 (glyceryl stearates (C_{18:0})), and m is a range of integers (e.g., 2 to 4, 2 to 6, 2 to 8, 2 to 10, 2 to 12, 2 to 14, 2 to 16, 4 to 6, 4 to 8, 4 to 10, 4 to 12, 4 to 14, 4 to 16, 6 to 8, 6 to 10, 6 to 12, 6 to 14, 6 to 16, 8 to 10, 8 to 12, 8 to 14, 8 to 16, 10 to 12, 10 to 14, 10 to 16, 12 to 14, 12 to 16, or 14 to 16).

Medium-chain glyceryl esters of a fatty acid include those having 6 to 12 carbons (e.g., such as m is 4, 6, 8, or 10). Exemplary medium-chain glyceryl esters include but are not limited to glyceryl caproates (C_{6:0}), glyceryl monocaprylate (C_{8:0}), glyceryl dicaprylate (C_{8:0}), glyceryl tricaprylate (C_{8:0}), glyceryl caprylate (C_{8:0})/caprate (C_{10:0}), glyceryl monocaprate (C_{10:0}), glyceryl dicaprate (C_{10:0}), glyceryl tricaprate (C_{10:0}), glyceryl monolaurate (C_{12:0}), glyceryl dilaurate (C_{12:0}), and glyceryl trilaurate (C_{12:0}). Long-chain glyceryl esters of a fatty acid include those having more than 12 carbons (e.g., such as m is 12 or higher). Exemplary long-chain glyceryl esters include but are not limited to glyceryl monomyristate (C_{14:0}), glyceryl dimyristate (C_{14:0}), glyceryl trimyristate (C_{14:0}), glyceryl palmitates (C_{16:0}), glyceryl stearates (C_{18:0}), glyceryl monooleate (C_{18:1}), glyceryl dioleate (C_{18:1}), glyceryl trioleate (C_{18:1}), glyceryl monolinoleate (C_{18:2}), glyceryl dilinoleate (C_{18:2}), and glyceryl trilinoleate (C_{18:2}).

Particular examples of glyceryl esters of a fatty acid are glyceryl monocaprylate, glyceryl dicaprylate, glyceryl monolaurate, and mixtures thereof. Capmul® MCM C8 (from Abitec Corp., Columbus, Ohio) is a commercially available mixture of glyceryl mono- and diesters having a minimum content of 90%-95% caprylic acid. Capmul® 708G (also from Abitec Corp.) is a commercially available glyceryl monoester having a minimum content of 90%-95% caprylic acid. Imwitor® 988 (from Sasol Germany GmbH, Witten, Germany) is a commercially available mixture of glyceryl mono-, di-, and triesters mainly having caprylic acid and a range of 47%-57% glyceryl monoesters. Additional examples include blends of glyceryl monocaprylate, glyceryl dicaprylate, or mixtures thereof with glyceryl monocaprate, glyceryl dicaprate, or mixtures thereof. Commercially available blends include Capmul® MCM (a mixture of glyceryl mono- and diesters mainly having caprylic acid and capric acid) and Imwitor® 742 (a mixture of glyceryl mono-, di-, and triesters mainly having caprylic acid and capric acid). Commercially available blends of glyceryl monolaurate include Lauroglycol® FCC and Laurglycol® 90.

In other examples, the lipophilic carrier can be a propylene glycol ester having the formula

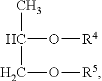

where R⁴ and R⁵ are, independently, H or C(O)—Y; each Y is, independently, optionally substituted C₄₋₂₄ alkyl or optionally substituted C₄₋₂₄ alkenyl; and at least one of R⁴ and R⁵ is C(O)—Y. Monoesters have one C(O)—Y group, where diesters have two such groups.

In some embodiments, C(O)—Y is C(O)—(CH₂)ₙ—CH₃, where n is, independently, an integer of from 2 to 12 and at least one of R⁴ and R⁵ is C(O)—(CH₂)ₙ—CH₃. Exemplary propylene glycol esters are mono- and diesters, where n is 2 (propylene glycol butyrates (C_{4:0})), 4 (propylene glycol caproates (C_{6:0})), 6 (propylene glycol caprylates (C_{8:0})), 8 (propylene glycol caprates (C_{10:0})), 10 (propylene glycol laurates (C_{12:0})), and 12 (propylene glycol myristates (C_{14:0})), and n is a range of integers (e.g., 2 to 4, 2 to 6, 2 to 8, 2 to 10, 2 to 12, 4 to 6, 4 to 8, 4 to 10, 4 to 12, 6 to 8, 6 to 10, 6 to 12, 8 to 10, 8 to 12, or 10 to 12).

Particular examples of propylene glycol esters are propylene glycol monocaprylate, propylene glycol ester dicaprylate, and mixtures thereof. Capryol® PGMC (from Gattefosse Canada Inc., Toronto, Canada) is a commercially available mixture of mono- and diesters having a minimum content of 90% caprylic acid and 60% monoester. Capryol® 90 (also from Gattefosse Canada Inc.) is a commercially available monoester having a minimum content of 90% caprylic acid. Additional examples are blends of propylene glycol monocaprylate, propylene glycol dicaprylate, or mixtures thereof with propylene glycol monocaprate, propylene glycol dicaprate, or mixtures thereof. Commercially available blends include Labrafac® PG (also available from Gattefosse Canada Inc.), which is a mixture of diesters mainly having caprylic acid and capric acid (HLB=2).

Additional exemplary lipophilic carriers are medium chain glyceryl triesters, such as caprylic/capric glyceryl triesters having 65%-80% caprylic/20%-35% capric fatty acids (Miglyol® 810N), 50%-65% caprylic/30%-45% capric fatty acids (Miglyol® 812N, HLB=2), <6% caproic/55%-85% caprylic/15%-40% capric/<4% lauric fatty acids (Captex® 300), and 50%-80% caprylic/20%-50% capric/<3% lauric fatty acids (Labrafac™ Lipophile WL 1349, HLB=2); medium chain glyceryl mono-, di-, and triesters, such as glyceryl monocaprylate, glyceryl dicaprylate, glyceryl tricaprylate, glyceryl caprylate/caprate, glyceryl monocaprate, glyceryl dicaprate, glyceryl tricaprate, glyceryl monolaurate, glyceryl dilaurate, glyceryl trilaurate, and mixtures thereof; long chain glyceryl mono-, di-, and triesters, and mixtures thereof, such as glyceryl monooleate having >60% C_{18:1}/<³5% C₁₈₋₂/<6% C₁₈₋₀ (Peceol®, HLB=3.3), glyceryl monolinoleate having 10-35% C_{18:1}/>50% C_{18:1}/<6% C_{18:0}/4%-20% C₁₆ (Maisine® 35-1, HLB=4), and glyceryl myristates; polyethoxylated glyceryl esters, such as PEG 6 oleyl glyceryl ester having 58%-80% C_{18:1}/15%-35% C_{18:2}/<6% C_{18:0}/4%-9% C₁₆ (Labrafil® M1944 CS, HLB=4) and PEG linoleyl glyceryl ester having 58%-80% C_{18:1}>15% C_{18:2}/<6% C_{18:0}/4%-9% C₁₆ (Labrafil® M2125 CS, HLB=4); propylene glycol esters, such as propylene glycol monocaprylate, propylene glycol dicaprylate, propylene glycol dicaprylocaprate (Labrafac™ PG), propylene glycol monocaprate, propylene glycol dicaprate, propylene glycol monolaurate, and propylene glycol dilaurate; medium-chain saturated fatty acids; ethyl oleate; and natural oils, such as olive oil, castor oil, coconut oil, corn oil, cottonseed oil, peanut oil, sesame oil, soybean oil, sunflower oil, and hydrolyzed forms thereof (e.g., hydrolyzed corn oil); and mixtures thereof.

Any of the lipophilic carriers described herein can be partially ethoxylated, where a free hydroxyl group is ethoxylated with ethylene glycol or ethylene oxide. Any useful polyethylene glycol groups can be used, such as a polyethylene glycol 300 having an average molecular weight of from about 300 to about 500 (equivalent from about 6 to about 8 moles of ethylene oxide).

Surfactant Carriers

The formulations of the invention can include one or more surfactant carriers. Surfactant carriers generally have a relatively high HLB (e.g., from 10 to 20). High HLB levels may be useful in promoting rapid formation of microemulsions and/or dispersions in an aqueous environment. In particular, the quantity and amphiphilic nature of the surfactant carrier can be tuned to dissolve or solubilize high levels of compound 1, compound 2, or a salt thereof, and to prevent precipitation at conditions expected in the gastrointestinal lumen (e.g., changes in pH in the median gastric pH or median duodenal pH in a fasted state or a fed state).

In some examples, the surfactant carrier can be a polyethoxylated ester of a fatty acid having the formula R⁶—C(O)O—(CH₂CH₂O)ₚ—R⁷, where R⁶ and R⁷ are, independently, H, optionally substituted C₁₂₋₂₄ alkyl, or optionally substituted C₁₂₋₂₄ alkenyl; p is an integer of from 5 to 50; and at least one of R⁶ or R⁷ is an optionally substituted C₁₂₋₂₄ alkyl or optionally substituted C₁₂₋₂₄ alkenyl. Optional substituents include one or more of C₁₋₃ alkyl, hydroxyl, or C₁₋₃ alkoxy. Exemplary polyethoxylated esters of a fatty acid are polyoxyl 40 stearate, polyoxyl 8 stearate, and PEG 15 hydroxystearate.

In other examples, the surfactant carrier can be a polyethoxylated alkyl ether having the formula R⁸—O—(CH₂CH₂O)_{q}—R⁹, where R⁸ and R⁹ are, independently, H, optionally substituted C₁₂₋₂₄ alkyl, or optionally substituted C₁₂₋₂₄ alkenyl; q is an integer of from 5 to 50; and at least one of R⁸ or R⁹ is an optionally substituted C₁₂₋₂₄ alkyl or optionally substituted C₁₂₋₂₄ alkenyl. Optional substituents include one or more of C₁₋₃ alkyl, hydroxyl, or C₁₋₃ alkoxy. Exemplary polyethoxylated alkyl ethers are polyoxyl 10 oleoyl ether and PEG 25 cetostearyl ether.

Exemplary surfactant carriers are liquid and solid polyethoxylated esters of fatty acids, such as polyoxyl 40 stearate (Myrj® 52, HLB=17), PEG 400 monostearate, also known as polyoxyl 8 stearate (Myrj® 45, HLB=11), and PEG 660 hydroxystearate, also known as PEG 15 hydroxystearate (Solutol® HS 15, HLB=14 to 16); polyethoxylated alkyl ethers, such as polyoxyl 10 oleoyl ether (Brij® 97 aka Brij® 96, HLB=12.4) and PEG 25 cetostearyl ether (Cremophor® A 25, HLB=15 to 17); polyethoxylated sorbitan esters, such as polysorbate 20 (Tween® 20, HLB=15) and polysorbate 80 (Tween® 80, HLB=15); polyethoxylated glyceryl esters having high HLB values (e.g., from 10 to 20), such as polyoxyl 35 castor oil (Cremophor® EL, HLB=12 to 14) and polyoxyl 40 castor oil having 40-45 moles of ethylene oxide (Cremophor® RH-40, HLB=14 to 16); polyethoxylated glyceryl esters of fatty acids having high HLB values (e.g., from 10 to 20), such as a mixture of PEG 6 caprylic/capric glyceryl esters having <2% C₆/50%-80% C₈/20%-50% C₁₀/<3% C₁₂/<1% C₁₄ (Softigen® 767, HLB=19), a mixture of PEG 8 caprylic/capric glyceryl esters having 50%-80% C₈/20%-50% C₁₀/<3% C₁₂/<1% C₁₈ (Labrasol®, HLB=14), a mixture of PEG 32 lauryl glyceryl esters having 40%-50% C₁₂/14%-24% C₁₄/4%-10% C₈/3-9% C₁₀/4%-14% C₁₆/5%-15% C₁₈ (Gelucire® 44/14, HLB=14), and a mixture of PEG 32 stearyl glyceryl esters having 40%-50% C₁₆/48%-58% C₁₈ (Gelucire® 50/13, HLB=13); polyethoxylated vitamin analogs, such as D-alpha-tocopheryl PEG 1000 succinate (HLB=13); and ethoxylated propoxylated block copolymers having formula H(OCH₂CH₂)ₐ(OCHCH₃CH₂)_{b}(OCH₂CH₂)ₐOH, where a is about 12 and b is about 20 (Poloxamer® 124), where a is about 38 and b is about 29, where a is about 80 and b is about 27 (Poloxamer® 188), where a is about 64 and b is about 37 (Poloxamer® 237), where a is about 141 and b is about 44 (Poloxamer® 338), where a is about 49 and b is about 57, and where a is about 101 and b is about 56 (Poloxamer® 407).

Any of the surfactant carriers described herein can be partially ethoxylated, where a free hydroxyl group is ethoxylated with ethylene glycol or ethylene oxide. Any useful polyethylene glycol groups can be used, such as a polyethylene glycol 300 having an average molecular weight of from about 300 to about 500 (equivalent from about 6 to about 8 moles of ethylene oxide).

### Co-Solvent Carriers

The formulations of the invention optionally include one or more co-solvent carriers. Generally, co-solvent carriers can aid in maintaining high surfactant carrier concentrations in a stable self-emulsifying state. Exemplary co-solvent carriers are ethanol, glycerol, propylene glycol (PG), polyethylene glycol (e.g., PEG 400, PEG 4000, or PEG 8000), propylene carbonate, diethylene glycol monoethyl ether (Transcutol®), glycofurol (tetrahydrofurfuryl alcohol polyethylene glycol ether), and N-methyl-2-pyrrolidone.

Crystallization Inhibiting Carriers

One or more crystallization inhibiting carriers can be included to optimize stability and/or promote self-emulsification of the composition. Exemplary crystallization inhibiting carriers include one or more of cellulose acetate phthalate (CAP), methylcellulose acetate phthalate, hydroxypropyl cellulose (HPC), hydroxypropylmethyl cellulose (HPMC), hydroxypropylmethyl cellulose acetate, hydroxypropylmethyl cellulose acetate succinate (HPMCAS), a polyvinyl pyrrolidone (PVP), a polyvinyl acetate (PVA), and a copolymer of a polyvinyl pyrrolidone and a polyvinyl acetate (PVP-PA) in any useful amount (e.g., from about 0.5% to about 5% (w/w), e.g., about 1% (w/w)). Further carriers are described below.

The crystallization inhibiting carriers can include one or more cellulose derivatives. Cellulose derivatives generally include those having any number of modifications to the free hydroxyl groups in cellulose. In some examples, the cellulose derivative is a cellulose acetate having from 10% to 50% acetyl. Referring to cellulose derivatives, % refers to the proportion of the free hydroxyl groups esterified with a functional group. For example, “10% acetyl” refers to a derivative having 10% of the free hydroxyl groups in cellulose esterified with an acetyl group.

Particular examples of cellulose acetates are cellulose acetate phthalates (CAP), such as those having 35% phthalyl, 24% acetyl (available as Cellacefate from Eastman Chemical Company, Kingsport, Tenn.); methylcellulose acetate phthalates;

hydroxypropylmethyl cellulose acetates; and hydroxypropylmethyl cellulose acetate succinates (HPMCAS), such as M grade having 9% acetyl/11% succinoyl (e.g., HPMCAS having a mean particle size of 5 μM (i.e., HPMCAS-MF, fine powder grade) or having a mean particle size of 1 mm (i.e., HPMCAS-MG, granular grade)), H grade having 12% acetyl/6% succinoyl (e.g., HPMCAS having a mean particle size of 5 μm (i.e., HPMCAS-HF, fine powder grade) or having a mean particle size of 1 mm (i.e., HPMCAS-HG, granular grade)), and L grade having 8% acetyl/15% succinoyl (e.g., HPMCAS having a mean particle size of 5 μm (i.e., HPMCAS-LF, fine powder grade) or having a mean particle size of 1 mm (i.e., HPMCAS-LG, granular grade)).

Additional exemplary cellulose derivatives are alkyl celluloses, such as methyl cellulose (Methocel™ A) or ethylcellulose (Ethocel®); hydroxyalkyl celluloses, such as hydroxymethyl cellulose, hydroxyethyl cellulose, hydroxypropyl cellulose (HPC, e.g., low-substituted HPC having 11% hydroxypropyl or 8% hydroxypropyl), and hydroxybutyl cellulose; hydroxyalkylalkyl celluloses, such as hydroxyethylmethyl cellulose and hydroxypropylmethyl cellulose (hypromellose, HPMC, e.g., those having about 19-24% methoxyl/7-12% hydroxypropxyl (Methocel™ K, including those having apparent viscosity (2% in water at 20° C.) of 80-120 cP (Methocel™ K100), 3,000-5,600 cP (Methocel™ K4M), 11,250-21,000 cP (Methocel™ K15M), 80,000-120,000 cP (Methocel™ K100M), available from Dow Chemical Co.), 28-30% methoxyl/7-12% hydroxypropxyl (Methocel™ E, including those having apparent viscosity (2% in water at 20° C.) of 3,000-5,600 cP (Methocel™ E4M) and 7,500-14,000 cP (Methocel™ E10M), also available from Dow Chemical Co.), 23% methoxyl/10% hydroxypropxyl (Metolose® SR, available from Shin-Etsu Chemical Co., Ltd., Tokyo, Japan), 23%-29% methoxyl/8%-9% hydroxypropxyl (Metolose®, also available from Shin-Etsu Chemical Co., Ltd.), 29% methoxyl/9% hydroxypropxyl (Hypromellose USP, substitution 2910), and 23% methoxyl/6% hydroxypropxyl (Hypromellose USP, substitution 2208)); hydroxyalkylalkyl cellulose esters, such as hydroxypropylmethyl cellulose phthalate (HPMCP) (e.g., HP 55 grade having 31% nominal phthalyl content and HP-55S or HP-50 grades having 24% nominal phthalyl content); carboxyalkyl celluloses, such as carboxymethyl cellulose and alkali metal salts thereof, such as sodium salts; carboxyalkylalkyl celluloses, such as carboxymethylethyl cellulose; and carboxyalkyl cellulose esters, such as carboxymethyl cellulose butyrate, carboxymethyl cellulose propionate, carboxymethyl cellulose acetate butyrate, and carboxymethyl cellulose acetate propionate.

The crystallization inhibiting carriers can include one or more polyvinyl pyrrolidones, polyvinyl acetates, or copolymers thereof. Exemplary polyvinyl pyrrolidones and polyvinyl acetates are polyvinyl pyrrolidones (e.g., povidone, PVP, or soluble povidone) having molecular weights of about 2,500 (Kollidon®12 PF, weight-average molecular weight between 2,000 to 3,000), about 9,000 (Kollidon®17 PF, weight-average molecular weight between 7,000 to 11,000), about 25,000 (Kollidon®25, weight-average molecular weight between 28,000 to 34,000), about 50,000 (Kollidon®30, weight-average molecular weight between 44,000 to 54,000), and about 1,250,000 (Kollidon®90 or Kollidon®90F, weight-average molecular weight between 1,000,000 to 1,500,000); polyvinyl acetate esters, such as polyvinyl acetate phthalate (PVAP); polyethylene glycol-polyvinyl acetate copolymers, such as polyethylene glycol-polyvinylcaprolactam-polyvinylacetate copolymer (Soluplus®); and polyvinylpyrrolidone-polyvinyl acetate copolymers (PVP-VA), such as those having a 60:40 ratio of N-vinyl-2-pyrrolidone to vinyl acetate (copovidone, also available as Kollidon® VA 64) and a 20:80 ratio of N-vinyl-2-pyrrolidone to vinyl acetate (Kollidon® SR).

Hydrophobic-Lipophilic Balance

The lipophilic carriers and surfactant carriers used in the formulations of the invention can be characterized by the hydrophobic-lipophilic balance (“HLB”). HLB generally provides the degree of hydrophobicity or lipophilicity for a given molecule. HLB can be determined by any useful method, including the formula HLB=20×Mh/M, where Mh is the molecular mass of the hydrophilic region of the molecule and M is the molecule mass of the molecule, and the formula HLB=7+Σᵢ Nᵢ^{h}−ΣᵢNᵢ^{l}, where i is the number of groups, Nᵢ^{h} is a value for each i^{th} hydrophilic group, and Nᵢ^{l} is the value for each i^{th} lipophilic group. Values of N^{h} and N^{l} depend on the type of hydrophilic and lipophilic group, respectively. Exemplary values for N^{h} include 38.7 for —SO₄Na, 21.1 for —CO₂K, 19.1 for —CO₂Na, 9.4 for tertiary amine N, 6.8 for ester (sorbitan ring), 2.4 for ester (free), 2.1 for —CO₂H, 1.9 for —OH (free), 1.3 for —O—, 0.5 for —OH (sorbitan ring), and 0.33 for —(CH₂CH₂O)—; and for M include −1.66 for benzyl, −0.475 for —CH—, —CH₂—, —CH₃, and ═CH—, and −0.13 for —(CH₂CH₂CH₂O)—.

### Methods of Preparing Self-Emulsifying Carriers Systems

The pharmaceutical composition including the self-emulsifying carrier can be made using any useful method. Generally, one or more self-emulsifying carriers (e.g., a lipophilic carrier, a surfactant carrier, and/or a co-solvent carrier) and compound 1, compound 2, or a salt thereof, are mixed to form a solution. Then, the resultant solution is heated to a solubilization temperature (e.g., from 40° C. to 80° C., e.g., about 45° C. or about 75° C.) for one or more of the components; mixed until the component(s) and compound 1, compound 2, or a salt thereof, are dissolved; and then cooled. The solution is then used to fill any one of the unit dosage forms described herein (e.g., a capsule). In particular embodiments, compound 1 or compound 2 is screened or sieved prior to mixing with one or more self-emulsifying carriers. The mixing step can be accomplished by any useful method, such as by agitation with shaker plate, sonication, stirring, or a combination thereof, and under any useful condition, e.g., at or above the solubilization temperature.

In particular embodiments, the solution includes a lipophilic carrier and a surfactant carrier. In some embodiments, addition of a co-solvent carrier can enhance solubility of compound 1 or compound 2. Thus, exemplary solutions include those having a lipophilic carrier, a surfactant carrier, and a co-solvent carrier. Exemplary co-solvent carrier includes any described herein, e.g., diethylene glycol monoethyl ether or N-methyl-2-pyrrolidone

The solution can optionally include one or more crystallization inhibiting carriers to optimize stability. Exemplary crystallization inhibiting carriers include any described herein, such as cellulose acetate phthalate (CAP), methylcellulose acetate phthalate, hydroxypropyl cellulose (HPC), hydroxypropylmethyl cellulose (HPMC), hydroxypropylmethyl cellulose acetate, hydroxypropylmethyl cellulose acetate succinate (HPMCAS), a polyvinyl pyrrolidone (PVP), a polyvinyl acetate (PVA), and a copolymer of a polyvinyl pyrrolidone and a polyvinyl acetate (PVP-PVA) in any useful amount (e.g., from about 0.5% to about 5% (w/w), e.g., about 1% (w/w)).

### Dosage and Administration

For administration to animal or human subjects, the dosage of compound 1, compound 2, or a salt thereof, is typically 0.1 to 15 mg/kg, more preferably 3 to 5 mg/kg. However, dosage levels can be highly dependent on the nature of the condition, drug efficacy, the condition of the patient, the judgment of the practitioner, and the frequency and mode of administration.

Compound 1, compound 2, or a salt thereof, is preferably provided in a therapeutically effective amount, which may be, for example, a daily amount of from 25 mg to 1,600 mg, more preferably 40 mg to 800 mg, and even more preferably 80 mg to 320 mg. In one embodiment, a pharmaceutical composition comprising a compound 1, compound 2, or a salt thereof, comprises a capsule, for example in unit dosage form having from 20 mg to 250 mg of compound 1, compound 2, or a salt thereof, (e.g., from 20 mg to 250 mg, such as from 20 mg to 30 mg, from 20 mg to 40 mg, from 20 mg to 50 mg, from 20 mg to 75 mg, from 20 mg to 100 mg, from 20 mg to 125 mg, from 20 mg to 150 mg, from 20 mg to 175 mg, from 20 mg to 200 mg, from 20 mg to 225 mg, from 30 mg to 40 mg, from 30 mg to 50 mg, from 30 mg to 75 mg, from 30 mg to 100 mg, from 30 mg to 125 mg, from 30 mg to 150 mg, from 30 mg to 175 mg, from 30 mg to 200 mg, from 30 mg to 225 mg, from 30 mg to 250 mg, from 40 mg to 50 mg, from 40 mg to 75 mg, from 40 mg to 100 mg, from 40 mg to 125 mg, from 40 mg to 150 mg, from 40 mg to 175 mg, from 40 mg to 200 mg, from 40 mg to 225 mg, from 40 mg to 250 mg, from 50 mg to 75 mg, from 50 mg to 100 mg, from 50 mg to 125 mg, from 50 mg to 150 mg, from 50 mg to 175 mg, from 50 mg to 200 mg, from 50 mg to 225 mg, from 50 mg to 250 mg, from 60 mg to 75 mg, from 60 mg to 100 mg, from 60 mg to 125 mg, from 60 mg to 150 mg, from 60 mg to 175 mg, from 60 mg to 200 mg, from 60 mg to 225 mg, from 60 mg to 250 mg, from 70 mg to 75 mg, from 70 mg to 100 mg, from 70 mg to 125 mg, from 70 mg to 150 mg, from 70 mg to 175 mg, from 70 mg to 200 mg, from 70 mg to 225 mg, from 70 mg to 250 mg, from 80 mg to 100 mg, from 80 mg to 125 mg, from 80 mg to 150 mg, from 80 mg to 175 mg, from 80 mg to 200 mg, from 80 mg to 225 mg, from 80 mg to 250 mg, from 90 mg to 100 mg, from 90 mg to 125 mg, from 90 mg to 150 mg, from 90 mg to 175 mg, from 90 mg to 200 mg, from 90 mg to 225 mg, from 90 mg to 250 mg, from 100 mg to 125 mg, from 100 mg to 150 mg, from 100 mg to 175 mg, from 100 mg to 200 mg, from 100 mg to 225 mg, and from 100 mg to 250 mg). These unit dosage forms can be administered to achieve any daily amount described herein, such as by administering one to five times daily (e.g., one, two, three, four, or five times daily).

### Unit Dosage Forms

For use as treatment of human and animal subjects, compound 1, compound 2, or a salt thereof, can be formulated as pharmaceutical or veterinary compositions. Depending on the subject to be treated, the mode of administration, and the type of treatment desired (e.g., prevention, prophylaxis, or therapy) the compounds are formulated in ways consonant with these parameters. A summary of such techniques is found in Remington: The Science and Practice of Pharmacy, 21st Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compound 1, compound 2, or a salt thereof, may be present in amounts totaling 1-95% by weight of the total weight of the composition. The composition including compound 1, compound 2, or a salt thereof, and a self-emulsifying carrier may be provided in a dosage form that is suitable for oral administration. Thus, the pharmaceutical composition may be in the form of, e.g., hard capsules (e.g., hard gelatin capsules or hard hydroxypropyl methylcellulose capsules), soft gelatin capsules, enteric coated hard gelatin capsules, enteric coated soft gelatin capsules, minicapsules, lozenges, gelcaps, dragees, solutions, emulsions, or suspensions. The compositions may be formulated according to conventional pharmaceutical practice.

In particular embodiments, compound 1, compound 2, or a salt thereof, and a carrier (e.g., a self-emulsifying carrier) are included in a capsule. Compound 1, compound 2, or a salt thereof, in combination with a carrier can be in any form, such as a liquid, a semi-solid suspension, or a solid suspension. The form of the compound 1, compound 2, or a salt thereof, can be determined based on dose. For example, a capsule filled with a self-emulsifying carrier in liquid form can be used for approximately 10-25% drug loading, and combinations of the self-emulsifying carrier in a semisolid or solid suspension can be used for approximately 40%-60% drug loading.

Exemplary unit dosage forms are hard capsules (e.g., hard gelatin capsules or hard hydroxypropyl methylcellulose capsules) and soft gelatin capsules. When soft gelatin capsules are used, it is preferred that when a composition contains a polyethylene glycol, the composition of the soft gelatin capsule shell contains a humectant, for example, sorbitol, to prevent brittleness of the soft gelatin capsule.

### Utility and Treatment of Conditions

Conditions that can be treated using the compositions or formulations described herein include pain (e.g., chronic or acute pain), epilepsy, Alzheimer's disease, Parkinson's disease, diabetes, cancer, sleep disorders, obesity, mood disorders, psychosis such as schizophrenia, tinnitus, amyotrophic lateral sclerosis, glaucoma, ischaemia, spasticity disorders, obsessive compulsive disorder, restless leg syndrome, Tourette syndrome, overactive bladder, renal disease, neuroprotection, and addiction. For example, the condition can be pain (e.g., neuropathic pain or post-surgery pain), epilepsy, migraine, Parkinson's disease, depression, schizophrenia, psychosis, or tinnitus.

Epilepsy as used herein includes but is not limited to partial seizures such as temporal lobe epilepsy, absence seizures, generalized seizures, and tonic/clonic seizures.

Cancer as used herein includes but is not limited to breast carcinoma, neuroblastoma, retinoblastoma, glioma, prostate carcinoma, esophageal carcinoma, fibrosarcoma, colorectal carcinoma, pheochromocytoma, adenocarcinoma, insulinoma, lung carcinoma, melanoma, and ovarian cancer.

Acute pain as used herein includes but is not limited to nociceptive pain and post-operative pain. Chronic pain includes, but is not limited to, neuropathic pain, peripheral neuropathic pain such as post-herpetic neuralgia, diabetic neuropathic pain, neuropathic cancer pain, HIV-associated neuropathy, erythromelalgia, failed back-surgery syndrome, trigeminal neuralgia, and phantom limb pain; central neuropathic pain such as multiple sclerosis related pain, Parkinson disease related pain, post-stroke pain, post-traumatic spinal cord injury pain, lumbosacral radiculopathy, cervical radiculopathy, brachial radiculopathy, and pain in dementia; musculoskeletal pain such as osteoarthritic pain and fibromyalgia syndrome; inflammatory pain such as rheumatoid arthritis, juvenile idiopathic arthritis, ankylosing spondylitis, psoriatic arthritis, inflammatory bowel disease, primary dysmenorrhea, and endometriosis; headache such as migraine, cluster headache, tension headache syndrome, facial pain, and headache caused by other diseases; visceral pain such as interstitial cystitis, irritable bowel syndrome, and chronic pelvic pain syndrome; and mixed pain such as lower back pain, neck and shoulder pain, burning mouth syndrome, and complex regional pain syndrome.

In treating osteoarthritic pain, joint mobility can also improve as the underlying chronic pain is reduced. Thus, use of compositions and formulations of the present invention to treat osteoarthritic pain includes use of such compositions or formulations to improve joint mobility in patients suffering from osteoarthritis or other conditions presenting with decreased joint mobility.

The compositions and formulations described herein can be tested for efficacy in any standard animal model of pain. Various models test the sensitivity of normal animals to intense or noxious stimuli (physiological or nociceptive pain). These tests include responses to thermal, mechanical, or chemical stimuli. Thermal stimuli usually involve the application of hot stimuli (typically varying between 42-55° C.) including, for example: radiant heat to the tail (the tail flick test), radiant heat to the plantar surface of the hindpaw (the Hargreaves test), the hotplate test, and immersion of the hindpaw or tail into hot water. Immersion in cold water, acetone evaporation, or cold plate tests may also be used to test cold pain responsiveness. Tests involving mechanical stimuli typically measure the threshold for eliciting a withdrawal reflex of the hindpaw to graded strength monofilament von Frey hairs or to a sustained pressure stimulus to a paw (e.g., the Ugo Basile analgesiometer). The duration of a response to a standard pinprick may also be measured. When using a chemical stimulus, the response to the application or injection of a chemical irritant (e.g., capsaicin, mustard oil, bradykinin, ATP, formalin, or acetic acid) to the skin, muscle joints, or internal organs (e.g., bladder or peritoneum) is measured.

In addition, various tests assess pain sensitization by measuring changes in the excitability of the peripheral or central components of the pain neural pathway. In this regard, peripheral sensitization (i.e., changes in the threshold and responsiveness of high threshold nociceptors) can be induced by repeated heat stimuli as well as the application or injection of sensitizing chemicals (e.g., prostaglandins, bradykinin, histamine, serotonin, capsaicin, or mustard oil). Central sensitization (i.e., changes in the excitability of neurons in the central nervous system induced by activity in peripheral pain fibers) can be induced by noxious stimuli (e.g., heat), chemical stimuli (e.g., injection or application of chemical irritants), or electrical activation of sensory fibers.

Various pain tests developed to measure the effect of peripheral inflammation on pain sensitivity can also be used to study the efficacy of the compositions (Stein et al., Pharmacol. Biochem. Behay. 31: 445-451, 1988; Woolf et al., Neurosci. 62:327-331, 1994). Additionally, various tests assess peripheral neuropathic pain using lesions of the peripheral nervous system. One such example is the “axotomy pain model” (Watson, J. Physiol. 231:41, 1973). Other similar tests include the SNL test which involves the ligation of a spinal segmental nerve (Kim and Chung, Pain 50: 355, 1992), the Seltzer model involving partial nerve injury (Seltzer, Pain 43: 205-18, 1990), the spared nerve injury (SNI) model (Decosterd and Woolf, Pain 87:149, 2000), chronic constriction injury (CCI) model (Bennett, Muscle Nerve 16:1040, 1993), tests involving toxic neuropathies such as diabetes (streptozocin model), pyridoxine neuropathy, taxol, vincristine, and other antineoplastic agent-induced neuropathies, tests involving ischemia to a nerve, peripheral neuritis models (e.g., CFA applied peri-neurally), models of post-herpetic neuralgia using HSV infection, and compression models.

In all of the above tests, outcome measures may be assessed, for example, according to behavior, electrophysiology, neurochemistry, or imaging techniques to detect changes in neural activity.

### EXAMPLES

### Example 1

### In Vitro Absorption of Compound 1

Absorption of particular drugs may be limited by biological factors, such as reduced cellular permeability in the intestine. Many cellular mechanisms can influence permeability, such as reduced passive paracellular and transcellular transport or increased active export by the efflux transporter protein P-glycoprotein. Thus, the use of self-emulsifying carriers may not be beneficial for drugs having limited cellular permeability, where methods to increase the solubilization of a drug may not increase its permeability in the gastrointestinal tract. Experiments were conducted to assess the apparent intestinal permeability of compound 1 using a Caco-2 cell model. Overall, the data herein suggest that compound 1 is not limited by permeability in the intestine, and formulations that promote delivery of compound 1 to the gastrointestinal tract may reduce food effects or increase bioavailability.

Briefly, Caco-2 cells were used as an in vitro model for predicting absorption through the intestinal epithelium. Confluent monolayers of Caco-2 cells were grown on collagen-coated, microporous, polycarbonate membranes (12-well Costar Transwell® plates) that are placed between two chambers. The apical side of the monolayer was exposed to the buffer solution in a first chamber, but the basolateral side of the monolayer adhered to microporous membranes fluidically connected to a second chamber. The dosing solution was added to the first chamber for measurements in the apical-to-basolateral direction (A-to-B) and to the second chamber for measurements in the basolateral-to-apical direction (B-to-A). Accordingly, for A-to-B measurements, the first chamber was the receiver chamber and the second chamber was the donor chamber; for B-to-A measurements, the first chamber was the donor chamber and the second chamber was the receiver chamber. The permeability assay buffer was Hanks Balanced Salt Solution containing 10 mM HEPES and 15 mM glucose at a pH of 7.4. The buffer in the receiver chamber also contained 1% bovine serum albumin. The dosing solution concentration was 1 μM of compound 1 in the assay buffer. All donor chambers were first pre-incubated for five minutes with dosing solution to attempt to saturate any non-specific binding sites on the device with test compound. After five minutes, the solution was removed and replaced with fresh dosing solution, and time was recorded as 0. Cell monolayers were dosed on the apical side/first chamber (for A-to-B measurements) or basolateral side/second chamber (for B-to-A measurements) and incubated at 37° C. with 5% CO₂ in a humidified incubator. At 30 and 60 minutes, aliquots were taken from the receiver chambers and replaced with fresh assay buffer. Samples were taken from the donor chamber at 0 and 60 minutes. Each experiment was performed in triplicate.

The apparent permeability (Pₐₚₚ), percent recovery, and efflux ratio were calculated as follows:

$${P}_{app} [ cm {s}^{- 1} ] = \frac{d {C}_{r}}{d t} \times \frac{{V}_{r}}{A \times {C}_{0}} , \\ Percent recovery [ \% ] = 100 \times \frac{( {V}_{r} \times {C}_{r , final} ) + ( {V}_{d} \times {C}_{d , final} )}{{V}_{d} \times {C}_{N}} , \\ and Efflux ratio = \frac{{P}_{app} ( B \text{-} to \text{-} A )}{{P}_{app} ( A \text{-} to \text{-} B )} ,$$

where

$$\frac{d {C}_{r}}{d t} [ μ M {s}^{- 1} ]$$

is the slope of the cumulative concentration in the receiver chamber as a function of time; V, [cm³] is the volume of the receiver chamber; V_{d} [cm³] is the volume of the donor chamber; A [cm²] is the area of the cell monolayer, which is estimated to be about 1.13 cm² for the 12-well Costar Transwell® plates; C₀[μM] is the measured concentration in the donor chamber at t=0 hours; C_{N} [μM] is the nominal concentration of the dosing solution; C_{r,final} [μM] is the cumulative concentration in the receiver chamber at the end of the incubation period; and C_{d,final} [μM] is the cumulative concentration in the donor chamber at the end of the incubation period.

Table 2 shows the apparent permeability for the apical-to-basolateral direction (A-to-B), the basolateral-to-apical direction (B-to-A), and cell-free condition. Compound 1 was classified as having a high permeability coefficient, due to an apparent permeability value Pₐₚₚ(A-to-B) more than 1.0×10⁻⁶ cm/s. Efflux of compound 1 was not considered significant, due to an efflux ratio of less than 3. Thus, absorption of compound 1 in humans is not expected to be permeability limited.

TABLE 2

| | | Percent | | | | | Efflux Ratio |
|---|---|---|---|---|---|---|---|
| | Initial Donor | Recovery | Pₐₚₚ (10⁻⁶ cm/s) | | | | (Pₐₚₚ (B-to-A)/Pₐₚₚ |
| Direction | Conc. (μM) | (%) | R1 | R2 | R3 | Avg. | (A-to-B)) |
| A-to-B | 0.69 | 28 | 4.37 | 4.31 | 6.78 | 5.15 | 0.8 |
| B-to-A | 0.59 | 37 | 5.04 | 3.48 | 4.45 | 4.32 | |
| Cell-free | 0.68 | 54 | 1.71 | 2.21 | 1.69 | 1.87 | |

### Example 2

### Kinetic Solubility and Physical Stability for Formulations Having One Carrier

Twenty-one carriers were tested for solubility and stability. Compound 1 was added to achieve the maximum concentration that was soluble at the appropriate solubilization temperature (sol. temp.), where achieved concentrations included <25 to 100 mg/g (mg of compound 1 per g of carrier). These data, and other results of the solubility experiments, are provided in Table 3, where tested formulations only included a single carrier. HLB indicates the hydrophobic-lipophilic balance. For the type of carrier, L indicates a lipophilic carrier, S indicates a surfactant carrier, and C indicates a co-solvent. Overall, these data indicated strong affinity of compound 1 for glyceryl mono- and diesters, polyethylene glycol (PEG) esters, PEG ethers, and propylene glycol (PG) esters.

TABLE 3

| | | Type | | Sol. | Conc. | Sonication |
|---|---|---|---|---|---|---|
| | | of | | Temp. | Achieved | Duration |
| Carrier | Brand Name | carrier | HLB | (° C.) | (mg/g) | (min) |
| Caprylic/capric glycerides | Imwitor ® 742 | L | 5.5 | 50 | ~100 | 60 |
| Glyceryl monocaprylate | Capmul ® MCM | L | 5.5 | 50 | ~100 | 60 |
| | C8 | | | | | |
| Polyoxyl 40 stearate | Myrj ® 52 | S | 17 | 80 | ~100 | 60 |
| Lauroyl macrogol 32 | Gelucire ® 44/14 | S | 14 | 50 | ~50 | 75 |
| glycerides | | | | | | |
| PEG (polyoxyl) 10 oleyl ether | Brij ® 96 aka | S | 12 | 50 | ~50 | 90 |
| | Brij ® 97 | | | | | |
| PEG 8 caprylic/capric | Labrasol ® | S | 14 | 30 | ~50 | Overnight |
| glycerides | | | | | | |
| PEG 8 stearate | Myrj ® 45 | S | 11 | 50 | ~50 | 45 |
| Propylene glycol | Capryol ® 90 | L | 6 | 30 | ~50 | 30 |
| monocaprylate | | | | | | |
| Diethylene glycol monoethyl | Transcutol ® | C | — | 30 | ~50 | 90 |
| ether | | | | | | |
| Caprylic/capric triglycerides | Labrafac ® | L | 2 | 30 | ~25 | 75 |
| | Lipophile WL | | | | | |
| | 1349 | | | | | |
| PEG (polyoxyl) 15 | Solutol ® HS15 | S | 15 | 50 | ~25 | 30 |
| hydroxystearate | | | | | | |
| Polyethylene glycol 400 | PEG 400 | C | — | 30 | ~25 | Overnight |
| Propylene glycol monolaurate | Lauroglycol ® | L | 4 | 30 | ~25 | 30 |
| | FCC | | | | | |
| Caprylic/capric triglycerides | Miglyol ® 812N | L | 2 | 30 | <25 | — |
| Castor oil | — | — | — | 30 | <25 | — |
| D-alpha-tocopheryl PEG | vitamin E TPGS | S | 13 | 50 | <25 | — |
| 1000 succinate | | | | | | |
| PEG 6 oleyl glycerides | Labrafil ® | L | 4 | 30 | <25 | — |
| | M1944 CS | | | | | |
| Poloxamer 124 | Lutrol ® L44 | S | 16 | 30 | <25 | — |
| Polyoxyl 35 castor oil | Cremophor ® EL | S | 13 | 30 | <25 | — |
| Propylene glycol | Labrafac ® PG | L | 2 | 30 | <25 | — |
| dicaprylocaprate | | | | | | |
| Olive oil | — | — | — | 30 | <25 | |

Table 4 provides the testing conditions for carrier stability at either 25° C./60% RH (relative humidity) or 40° C./75% RH in 4 mL glass vials. Concentrations are provided in for concentration of compound 1 per concentration of carrier (mg/g).

TABLE 4

| | Concentrations |
|---|---|
| | monitored on |
| Carrier | stability (mg/g) |
| Caprylic/capric glycerides (Imwitor 742) | 100, 50, 25 |
| Glyceryl monocaprylate | 100, 50, 25 |
| Polyoxyl 40 stearate | 100, 50, 25 |
| PEG 8 stearate | 50, 25 |
| PEG (polyoxyl) 10 oleyl ether | 50, 25 |
| Lauroyl macrogol 32 glycerides | 50, 25 |
| Propylene glycol monocaprylate | 50, 25 |
| PEG 8 caprylic/capric glycerides | 50, 25 |
| Caprylic/capric triglycerides (Labrafac) | 25 |
| Propylene glycol monolaurate | 25 |
| PEG 15 hydroxystearate | 25 |
| Polyethylene glycol 400 | 25 |
| Diethylene glycol monoethyl ether | 50 |

Table 5 provides the maximum concentration of compound 1 per concentration of carrier (mg/g) achieved without precipitation after 1 week and 1 month in storage. Overall, these data show that various carriers provided formulations that were stable after one month.

TABLE 5

| | Physical Stability at | | | Physical Stability at | | |
|---|---|---|---|---|---|---|
| | 25° C./60% RH | | | 40° C./75% RH | | |
| | Conc. | | | Conc. | one | |
| Carrier | (mg/g) | one week | one month | (mg/g) | week | one month |
| Caprylic/capric glycerides (Imwitor 742) | 50 | >1 week | >1 month | 50 | >1 week | >1 week |
| Glyceryl monocaprylate | 50 | >1 week | >1 month | 50 | >1 week | >1 week |
| Polyoxyl 40 stearate | 50 | >1 week | >1 month | 50 | >1 week | >1 week |
| Lauroyl macrogol 32 glycerides | 25 | >1 week | <1 month | 25 | >1 week | >1 week |
| PEG (polyoxyl) 10 oleyl ether | 25 | >1 week | >1 month | 50 | >1 week | >1 month |
| PEG 8 caprylic/capric glycerides | 50 | >1 week | >1 month | 50 | >1 week | >1 month |
| PEG 8 stearate | 25 | >1 week | >1 month | 50 | >1 week | <1 month |
| Propylene glycol monocaprylate | 50 | >1 week | >1 month | 50 | >1 week | >1 month |
| Caprylic/capric triglycerides (Labrafac) | 25 | >1 week | >1 month | 25 | >1 week | >1 month |
| PEG 15 hydroxystearate | 25 | >1 week | >1 month | 25 | >1 week | >1 month |
| Polyethylene glycol 400 | 25 | >1 week | >1 month | 25 | >1 week | >1 month |
| Propylene glycol monolaurate | 25 | >1 week | >1 month | 25 | >1 week | <1 month |
| Diethylene glycol monoethyl ether | 50 | >1 week | >1 month | 50 | >1 week | >1 month |

### Example 3

### Dilutability Tests for Formulations Having One Carrier

Experiments were conducted to test the dilutability of formulations including a single carrier. Compound 1 was included at test concentrations of 25 to 50 mg of compound 1 per g of carrier (mg/g). These data, and other results of the dilutability experiments, are provided in Table 6. Dilutability experiments were conducted at a pH of 1.2, where caprylic/capric glycerides, glyceryl monocaprylate, propylene glycol monocaprylate, and polyoxyl 40 stearate provided the highest stable concentrations. As glyceryl monocaprylate and propylene glycol monocaprylate are both lipophilic carriers, these carriers were chosen as the main components for binary formulations having two carriers.

TABLE 6

| | Conc. | Placebo | Compound 1 |
|---|---|---|---|
| | Tested | Dilution | Dilution |
| Carrier | (mg/g) | (t = 6 h) | (t = 6 h^{#}) |
| Caprylic/capric glycerides | 50 | clear with | clear with some |
| (Imwitor 742) | | some oil | oil on top^{a} |
| | | on top | |
| Glyceryl monocaprylate | 50 | clear with | clear with some |
| | | some oil | oil on top^{a} |
| | | on top | |
| polyoxyl 40 stearate | 50 | clear | clear^{a} |
| PEG (polyoxyl) 10 oleyl ether | 50 | clear | clear^{a} |
| PEG 8 caprylic/capric glycerides | 50 | cloudy | cloudy; crystals |
| PEG 8 stearate | 25 | cloudy | cloudy^{a} |
| Propylene glycol monocaprylate | 50 | clear with | clear with some |
| | | some oil | oil on top^{a} |
| | | on top | |
| Caprylic/capric triglycerides | 25 | clear | clear with some |
| (Labrafac) | | | oil on top^{a} |
| PEG 15 hydroxystearate | 25 | clear | clear^{a} |
| Polyethylene glycol 400 | 25 | clear | turbid^{b} |
| Diethylene glycol monoethyl | 50 | clear | turbid^{b} |
| ether | | | |

^{#}unless otherwise specified
^{a}no crystals visible on microscope at 6 h
^{b}with compound precipitation upon dilution from the initial time point

### Example 4

### Phase Diagrams for Binary Formulations Having Two Carriers

Binary formulations having two carriers (a lipophilic carrier and a surfactant carrier) were investigated to potentially increase the concentration of compound 1 in the formulation and to decrease food effect. For these phase diagrams, compound 1 was not included. Formulations can be tested at various pHs to mimic the fasted state and fed state. Ternary phase diagrams were plotted for six binary lipophilic carrier/surfactant carrier combinations.

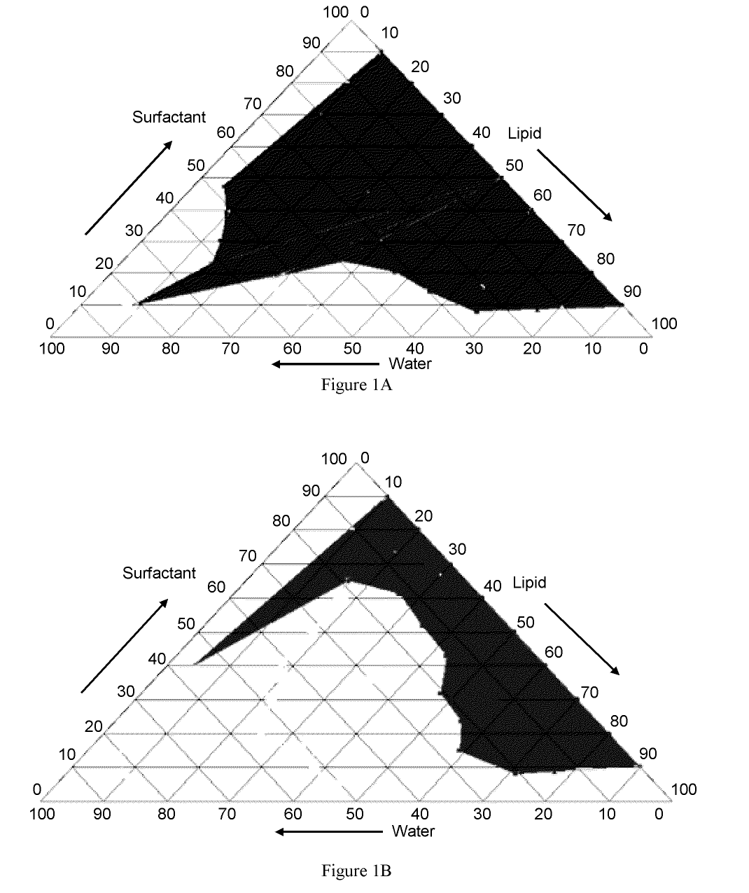

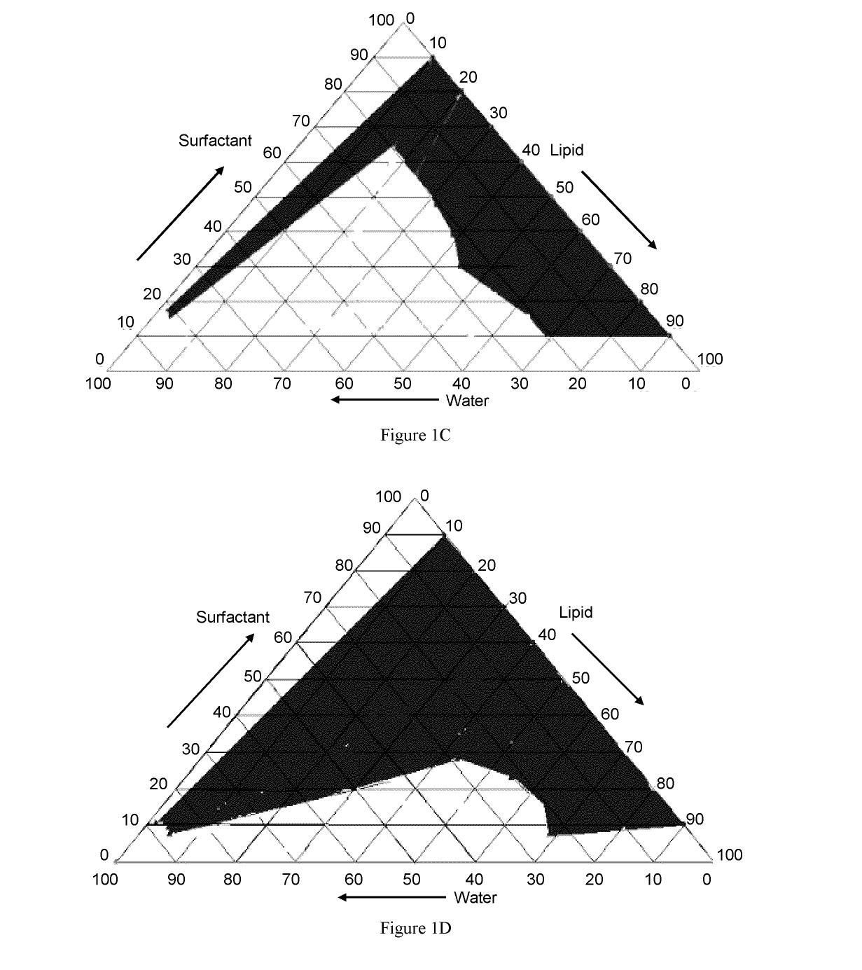

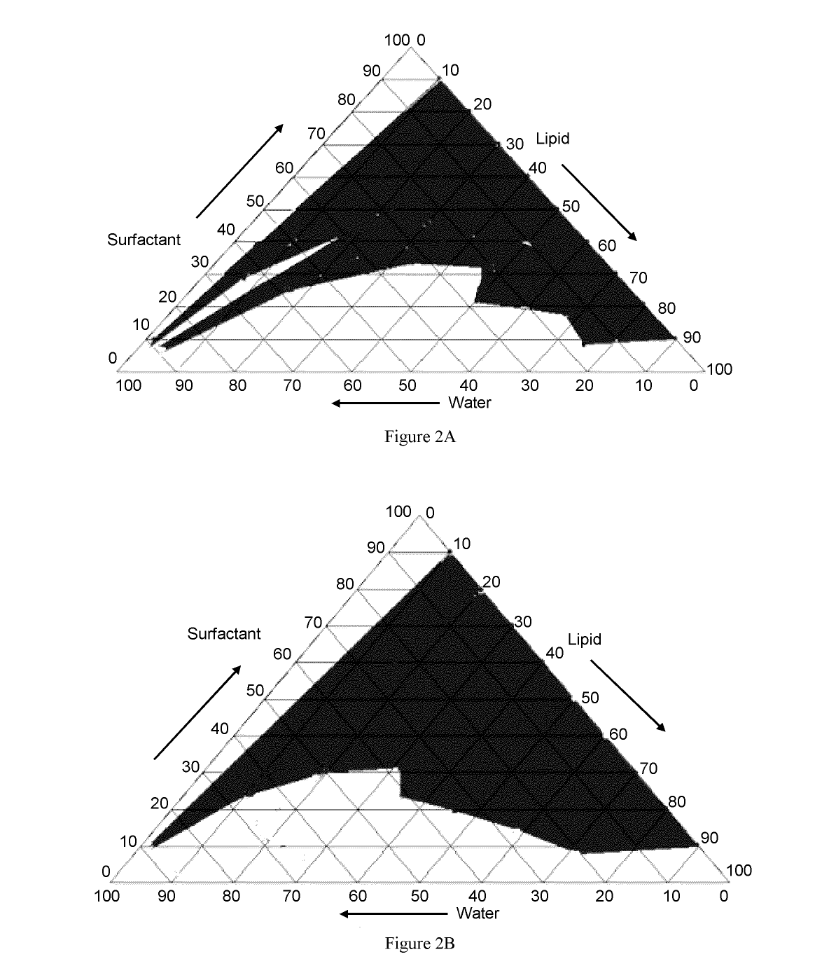

FIGS. 1A-1D show phase diagrams for lipophilic carrier glyceryl monocaprylate and surfactant carrier PEG 10 oleoyl ether (FIG. 1A), PEG 8 stearate (FIG. 1B), PEG 15 hydroxystearate (FIG. 1C), or polyoxyl 40 stearate (FIG. 1D). FIGS. 2A-2B are phase diagrams for lipophilic carrier propylene glycol monocaprylate and surfactant carrier PEG 10 oleoyl ether (FIG. 2A) and polyoxyl 40 stearate (FIG. 2B). Microemulsion regions were characterized as regions having mixtures that were optically clear or translucent, and these regions are shown in dark gray in FIGS. 1A-2B. Outside of the microemulsion regions, the binary formulations were cloudy. In particular, at any dilution with aqueous phase, from 0% to 90%, the binary formulation remained a microemulsion.

### Example 5

### Kinetic Solubility, Physical Stability, and Dilutability for Binary Formulations

Formulations A-E were selected for further testing. Compound 1 was added to achieve the maximum concentration that was soluble at the appropriate solubilization temperature (generally between 50° C. to 80° C.), where achieved concentrations included 50 to 105 mg/g (mg of compound 1 per g of carriers). These data, and results of the stability experiments, are provided in Table 7 for various binary formulations. Formulations B, E1, and E2 were stable for more than 1 month.

TABLE 7

| | | Conc. | Physical Stability | |
|---|---|---|---|---|
| | | Achieved | 25° C./ | 40° C./ |
| | Carriers | (mg/g) | 60% RH | 75% RH |
| A | 50/50 Glyceryl | 70 | <1 month | >1 month |
| | monocaprylate/ | | | |
| | PEG 10 oleoyl | | | |
| B | 20/80 Glyceryl | 50 | >1 month | >1 month |
| | monocaprylate/ | | | |
| | PEG 15 | | | |
| | hydroxystearate | | | |
| C | 30/70 Glyceryl | 150 | <1 month | <1 month* |
| | monocaprylate/ | | | |
| | Polyoxyl 40 | | | |
| | stearate | | | |
| D | 20/80 Propylene glycol | 150 | <1 month | <1 month |
| | monocaprylate/Polyoxyl | | | |
| | 40 stearate | | | |
| E | 20/80 Propylene glycol | 100 | <1 month** | <1 month** |
| | monocaprylate/PEG | | | |
| | 10 oleoyl ether | | | |
| E1 | 30/70 Propylene glycol | 80 | >1 month | >1 month |
| | monocaprylate/PEG | | | |
| | 10 oleoyl ether | | | |
| E2 | 30/70 Propylene glycol | 70 | >1 month | >1 month |
| | monocaprylate/PEG | | | |
| | 10 oleoyl ether | | | |

*for these carriers, the 65 mg/g sample was stable at one month
**these samples were stable at 80 mg/g at one month

Table 8 provides dilutability results for various binary formulations at pH of 1.2.

Precipitation of compound 1 was not observed for formulations B or E2 at 6 hours. Concentration tested (conc. tested) refers to the concentration of compound 1 per concentration of the carriers (mg/g).

TABLE 8

| | | | | | Compound 1 |
|---|---|---|---|---|---|
| | | Conc. | Placebo | Compound 1 | Dilution |
| | | tested | Dilution | Dilution | (t = 1 h, 3 h, 6 h, or |
| | Carriers | (mg/g)* | (t = 0) | (t = 0) | 24 h) |
| A | 50/50 Glyceryl | 70 | translucent | cloudy | cloudy; crystals |
| | monocaprylate/PEG 10 oleoyl | | | | (t = 6 h and 24 h) |
| B | 20/80 Glyceryl | 50 | translucent | translucent | translucent; |
| | monocaprylate/PEG 15 | | | | crystals (t = 24 h) |
| | hydroxystearate | | | | |
| C | 30/70 Glyceryl | 65 | transparent | translucent | translucent; |
| | monocaprylate/Polyoxyl 40 | | | | crystals (t = 3 h, 6 h, |
| | stearate | | | | and 24 h) |
| D | 20/80 Propylene glycol | 70 | transparent | cloudy | cloudy; crystals |
| | monocaprylate/Polyoxyl 40 | | | | (t = 1 h and 3 h)^{a} |
| | stearate | | | | |
| E1 | 20/80 Propylene glycol | 80 | cloudy | cloudy | cloudy; crystals |
| | monocaprylate/PEG 10 oleoyl | | | | (t = 3 h, 6 h, and 24 h) |
| | ether | | | | |
| E2 | 20/80 Propylene glycol | 70 | cloudy | cloudy | cloudy; no crystals |
| | monocaprylate/PEG 10 oleoyl | | | | observed up to 24 h |
| | ether | | | | |

^{a}Indicates that the test was discontinued after 3 hours
*“Conc. tested” provides concentrations having one month stability at both 25° C./60% RH and 40° C./75% RH

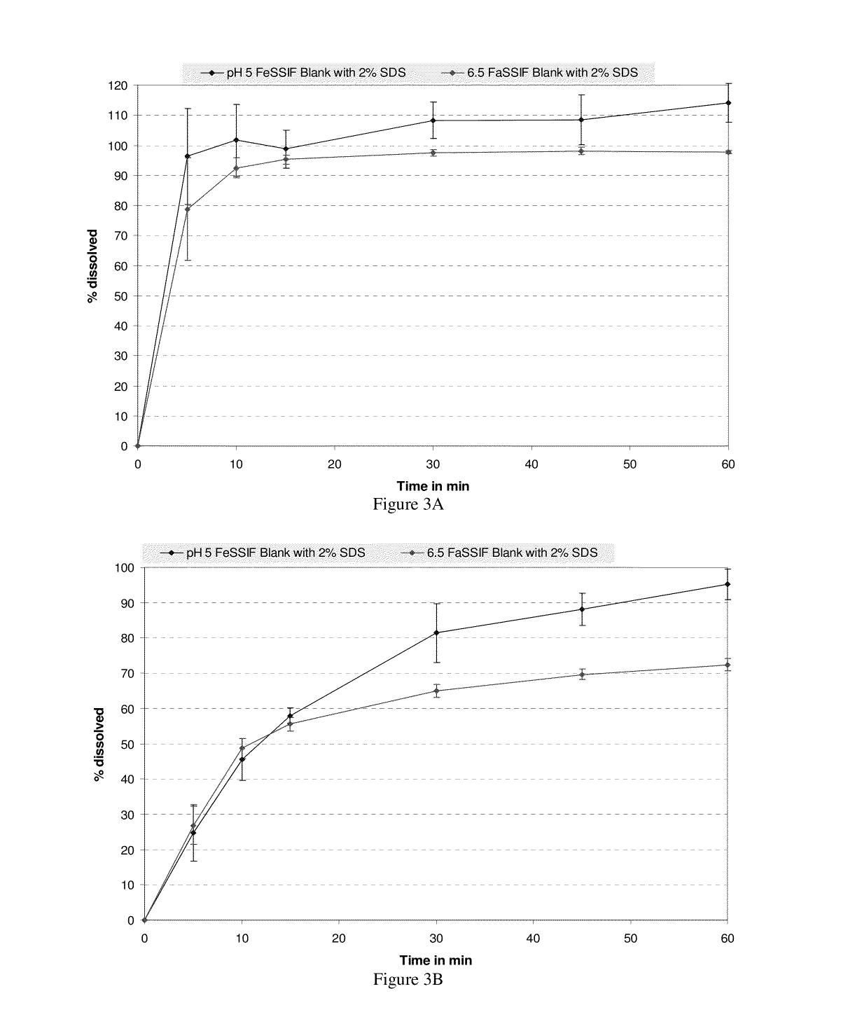

Formulations B and E2 were further tested at pH 5 and 6.8, which represent the pH of the fed and fasted states, respectively, in the small intestine. The objective of these dilutability tests was to evaluate the effect of these pH values on the formulation. Generally, changes in dilution behavior based on pH would anticipate a greater food effect. Conversely, a formulation having similar dilution behavior for pH of 5 and 6.8 would be expected to have minimal food effect. Table 9 shows that the formulations have similar dilution behavior at the different tested pH values of 5 and 6.8. Concentration tested (conc. tested) refers to the concentration of compound 1 per concentration of the carriers (mg/g). As shown in FIGS. 3A-3B, both formulations exhibited similar dissolution profiles in FeSSIF and FaSSIF at pH of 5 and of 6.5.

TABLE 9

| | | Conc. | | Placebo | Compound 1 | |
|---|---|---|---|---|---|---|
| | | tested | | Dilution | Dilution | Compound 1 Dilution |
| | Carriers | (mg/g) | pH | (t = 0) | (t = 0) | (t = 1 h, 3 h, 6 h, or 24 h) |
| B | 20/80 Glyceryl | 50 | 5 | transparent | cloudy with particles | cloudy; crystals (t = 3 h, |
| | monocaprylate/ | | | | visible on top of | 6 h, and 24 h) |
| | PEG 15 | | | | liquid | |
| | hydroxystearate | | 6.8 | transparent | cloudy with particles | cloudy; crystals (t = 1 h, |
| | | | | | visible on top of | 3 h, 6 h, and 24 h) |
| | | | | | liquid | |
| E2 | 20/80 Propylene | 70 | 5 | cloudy | cloudy | cloudy; crystals (t = 1 h, |
| | glycol | | | | | 3 h, 6 h, and 24 h) |
| | monocaprylate/ | | 6.8 | cloudy | cloudy | cloudy; crystals (t = 1 h, |
| | PEG 10 oleoyl | | | | | 6 h, and 24 h) |
| | ether | | | | | |

### Example 6

### In Vivo Study in Rats

The pharmacokinetics of formulations B (50 mg/g of compound 1/carrier) and E2 (70 mg/g of compound 1/carrier) were studied in rats. Table 10 provides a summary of the results for this in vivo study. Control indicates compound 1 in 0.5% Tween® 80 in 0.5% carboxy methylcellulose (CMC). Doses included 10 to 100 mg of compound 1 to kg of the subject (mg/kg).

TABLE 10

| | | | | | AUC_{∞} | AUC₀₋ₗₐₛₜ |
|---|---|---|---|---|---|---|
| | | Cₘₐₓ | Tₘₐₓ | t_{1/2} | (h * | (h * |
| | For- | (ng/mL, | (hr, | (hr, | mg/mL, | mg/mL, |
| Dose | mulation | mean) | mean) | mean) | mean) | mean) |
| 10 mg/kg | Control | 89 | 3.33 | 1.54 | 503 | 466 |
| dose | B | 368 | 2.50 | 2.67 | 1623 | 1326 |
| | E2 | 150 | 6.67 | 2.08 | 978 | 723 |
| 30 mg/kg | Control | 152 | 3.33 | 4.07 | 1262 | 831 |
| dose | B | 964 | 1.00 | 6.20 | 7458 | 4019 |
| | E2 | 206 | 8.00 | n/a | n/a | 982 |
| 100 mg/kg | Control | 450 | 5.33 | 3.22 | 3461 | 2155 |
| dose | B | 1597 | 0.833 | 4.21 | 8626 | 6193 |
| | E2 | 194 | 3.00 | 6.74 | 1795 | 819 |

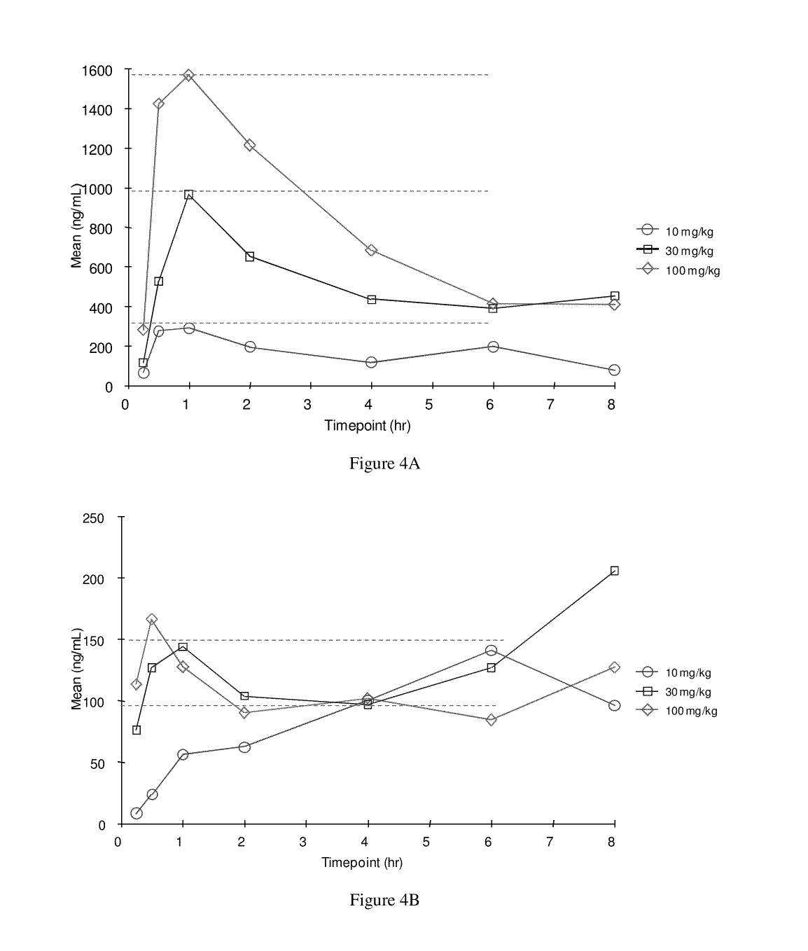

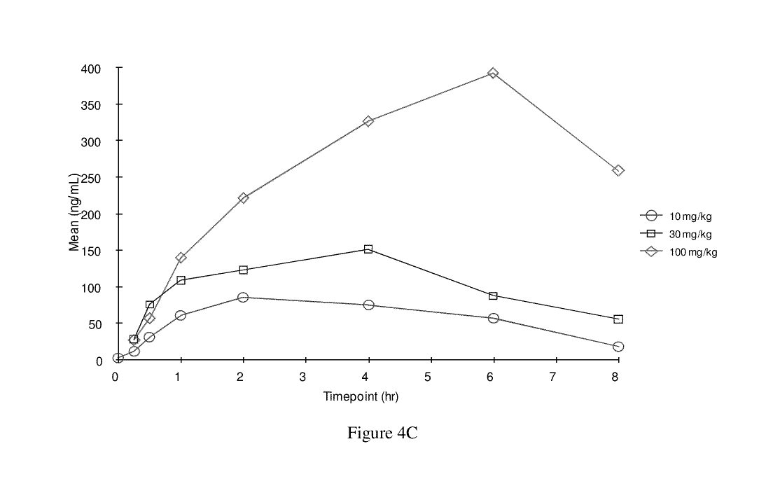

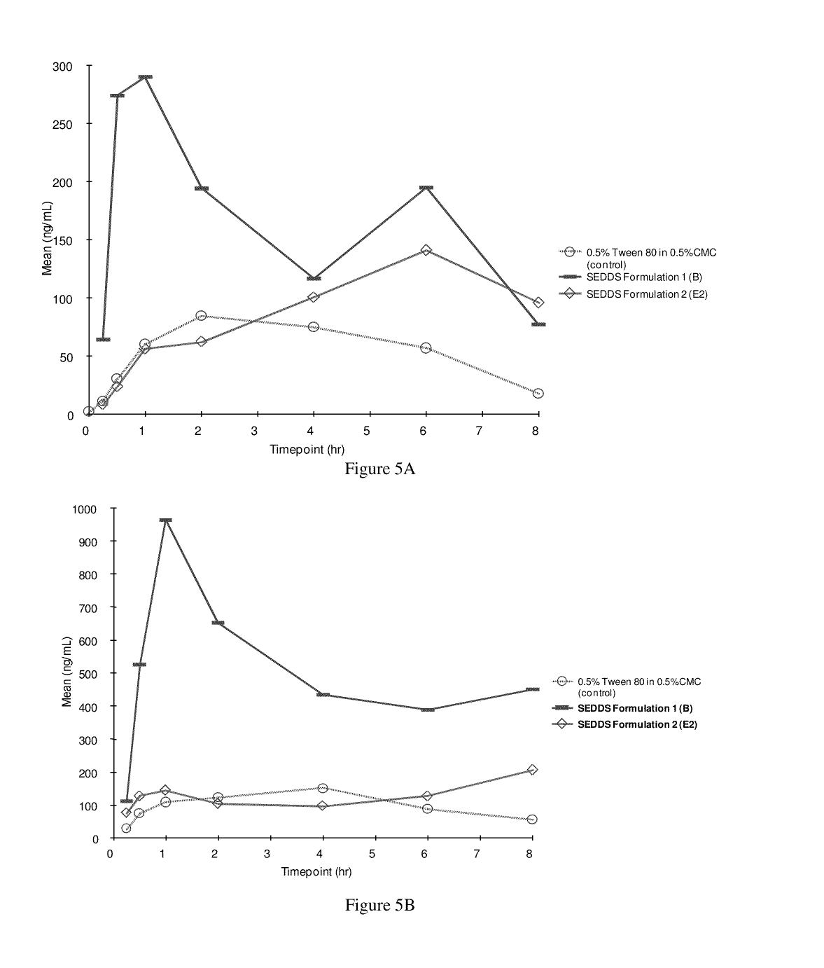

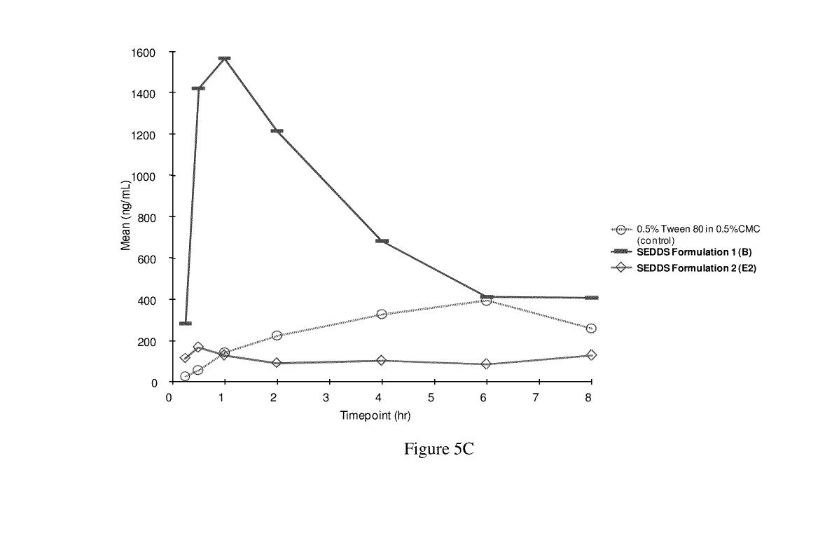

Data for each animal are provided for formulation B (Table 11 and FIG. 4A), formulation E2 (Table 12 and FIG. 4B), and control (Table 13 and FIG. 4C) for three doses. FIGS. 5A-5C provides data for these formulations for each dose. Overall, formulation B showed enhanced oral bioavailability of compound 1, compared to control.

TABLE 11

| Dose of | | Pharmacokinetic parameters | | | | |
|---|---|---|---|---|---|---|
| Formulation | | | | | | AUC₀₋ₗₐₛₜ |
| B | | Cₘₐₓ | Tₘₐₓ | t_{1/2} | AUC_{∞} | (h * |
| (50 mg/g) | Animal | (ng/mL) | (hr) | (hr) | (h * ng/mL) | ng/mL) |
| 10 mg/kg | 28 | 302 | 0.50 | 4.89 | 1810 | 1211 |
| | 29 | 449 | 1.00 | 1.78 | 1296 | 1244 |
| | 30 | 353 | 6.00 | 1.34 | 1763 | 1522 |
| | Mean | 368 | 2.50 | 2.67 | 1623 | 1326 |
| | SD | 74.6 | 3.04 | 1.94 | 284 | 171 |
| | SEM | 43.1 | 1.76 | 1.12 | 164 | 98.7 |
| 30 mg/kg | 31 | 874 | 1.00 | 8.55 | 7807 | 3650 |
| | 32 | 679 | 1.00 | NC | NC | 3555 |
| | 33 | 1340 | 1.00 | 3.85 | 7109 | 4851 |
| | Mean | 964 | 1.00 | 6.20 | 7458 | 4019 |
| | SD | 340 | 0.00 | 3.3 | 494 | 723 |
| | SEM | 196 | 0.00 | 2.4 | 349 | 417 |
| 100 mg/kg | 34 | 1720 | 1.00 | 4.77 | 12953 | 8708 |
| | 35 | 1590 | 1.00 | 2.73 | 7174 | 5537 |
| | 36 | 1480 | 0.500 | 5.12 | 5752 | 4334 |
| | Mean | 1597 | 0.833 | 4.21 | 8626 | 6193 |
| | SD | 120 | 0.289 | 1.29 | 3814 | 2260 |
| | SEM | 69.4 | 0.167 | 0.745 | 2202 | 1305 |

TABLE 12

| Dose of | | Pharmacokinetic parameters | | | | |
|---|---|---|---|---|---|---|
| Formulation | | | | | | AUC₀₋ₗₐₛₜ |
| E2 | | Cₘₐₓ | Tₘₐₓ | t_{1/2} | AUC_{∞} | (h * |
| (70 mg/g) | Animal | (ng/mL) | (hr) | (hr) | (h * ng/mL) | ng/mL) |
| 10 mg/kg | 37 | 158 | 6.00 | 1.38 | 796 | 680 |
| | 38 | 158 | 6.00 | 2.78 | 1161 | 776 |
| | 39 | 133 | 8.00 | NC | NC | 713 |
| | Mean | 150 | 6.67 | 2.08 | 978 | 723 |
| | SD | 14.4 | 1.15 | 0.985 | 258 | 48.9 |
| | SEM | 8.33 | 0.667 | 0.697 | 182 | 28.2 |
| 30 mg/kg | 40 | 262 | 8.00 | NC | NC | 1382 |
| | 41 | 131 | 8.00 | NC | NC | 674 |
| | 42 | 224 | 8.00 | NC | NC | 891 |
| | Mean | 206 | 8.00 | n/a | n/a | 982 |
| | SD | 67.4 | 0.00 | n/a | n/a | 363 |
| | SEM | 38.9 | 0.00 | n/a | n/a | 210 |
| 100 mg/kg | 43 | 255 | 0.500 | 6.17 | 2006 | 902 |
| | 44 | 174 | 8.00 | NC | NC | 841 |
| | 45 | 154 | 0.500 | 7.30 | 1583 | 715 |
| | Mean | 194 | 3.00 | 6.74 | 1795 | 819 |
| | SD | 53.5 | 4.33 | 0.796 | 299 | 95.5 |
| | SEM | 30.9 | 2.50 | 0.563 | 211 | 55.1 |

TABLE 13

| | | Pharmacokinetic parameters | | | | |
|---|---|---|---|---|---|---|
| | | | | | | AUC₀₋ₗₐₛₜ |
| Dose of | | Cₘₐₓ | Tₘₐₓ | t_{1/2} | AUC_{∞} | (h * |
| Control | Animal | (ng/mL) | (hr) | (hr) | (h * ng/mL) | ng/mL) |
| 10 mg/kg | 1 | 96.7 | 6.00 | 0.858 | 558 | 534 |
| | 2 | 110 | 2.00 | 1.65 | 559 | 517 |
| | 3 | 60.6 | 2.00 | 2.13 | 391 | 346 |
| | Mean | 89 | 3.33 | 1.54 | 503 | 466 |
| | SD | 26 | 2.3 | 0.64 | 97.0 | 104 |
| | SEM | 15 | 1.3 | 0.37 | 56.0 | 60.0 |
| 30 mg/kg | 4 | 125 | 2.00 | 8.28 | 1888 | 845 |
| | 5 | 169 | 4.00 | 1.34 | 898 | 857 |
| | 6 | 162 | 4.00 | 2.59 | 998 | 790 |
| | Mean | 152 | 3.33 | 4.07 | 1262 | 831 |
| | SD | 23.6 | 1.15 | 3.70 | 545 | 35.3 |
| | SEM | 13.7 | 0.667 | 2.14 | 315 | 20.4 |
| 100 mg/kg | 7 | 434 | 4.00 | 2.88 | 2812 | 2121 |
| | 8 | 361 | 6.00 | 2.49 | 2637 | 1893 |
| | 9 | 554 | 6.00 | 4.29 | 4934 | 2453 |
| | Mean | 450 | 5.33 | 3.22 | 3461 | 2155 |
| | SD | 97.4 | 1.15 | 0.945 | 1279 | 282 |
| | SEM | 56.3 | 0.667 | 0.545 | 738 | 163 |

### Example 7

### In Vivo Study in Dogs

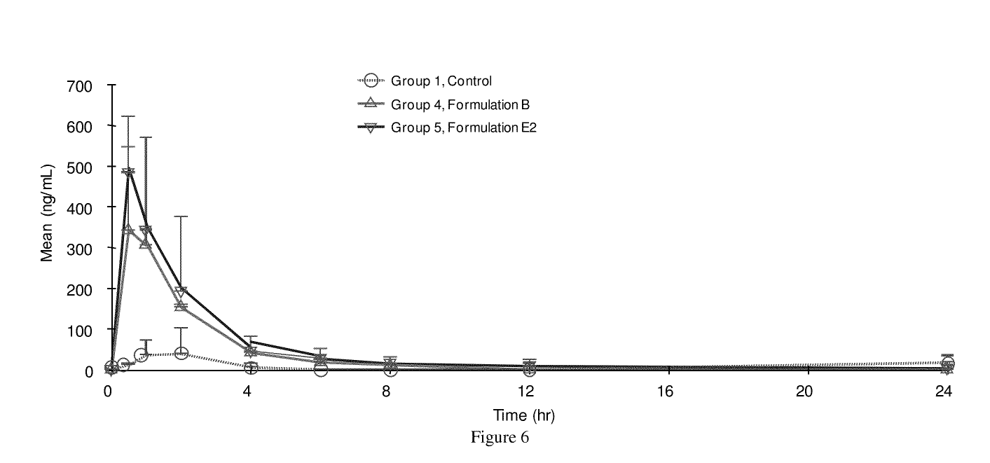

The pharmacokinetics of formulations B and E2 were studied in dogs using a dosage of 10 mg/kg (mg of compound 1/kg of subject). Table 14 provides a summary of the results for this in vivo study. Control indicates compound 1HCl in 0.5% Tween 80 in 0.5% CMC. FIG. 6 shows enhanced oral bioavailability for formulations B and E2.

TABLE 14

| | | | Tₘₐₓ | t_{1/2} | Cₘₐₓ | AUC₀₋ₗₐₛₜ | AUC₀₋₂₄ | AUC_{∞} |
|---|---|---|---|---|---|---|---|---|
| | Animal | Rsq | (hr) | (hr) | (ng/mL) | (hr * ng/mL) | (hr * ng/mL) | (hr * ng/mL) |
| Group 1 | 1311019 | NC | 1 | NC | 14.4 | 7.82 | 15.0 | NC |
| (control) | 1311108 | 0.03 | 2 | NR | 111 | 470 | 470 | NR |
| | 1457366 | NC | 1 | NC | 17.3 | 131 | 131 | NC |
| | Mean | 0.03 | 1.3 | NC | 47.6 | 203 | 205 | NC |
| | SD | NA | 0.6 | NA | 55 | 239 | 236 | NA |
| Group 4 | 1177436 | 0.99 | 0.5 | 2 | 486 | 864 | 885 | 894 |
| (formulation | 1250991 | 0.95 | 1 | 1.4 | 276 | 651 | 674 | 675 |
| B) | Mean | 0.97 | 0.8 | 1.7 | 381 | 758 | 779 | 784 |
| | SD | 0.03 | NA | NA | NA | NA | NA | NA |
| Group 5 | 1311655 | 1 | 0.5 | 8.8 | 640 | 2000 | 2000 | 2120 |
| (formulation | 1394152 | 0.96 | 0.5 | 1.5 | 408 | 534 | 550 | 569 |
| E2) | 1485530 | 0.86 | 0.5 | 1.4 | 405 | 611 | 635 | 635 |
| | Mean | 0.94 | 0.5 | 3.9 | 484 | 1050 | 1060 | 1110 |
| | SD | 0.07 | 0 | 4.3 | 135 | 824 | 813 | 875 |

NC = Not calculated by WinNonlin due to insufficient data points for elimination phase.
NR = Not reported, due to poor goodness-of-fit (R² < 0.8) for elimination phase.

In Table 14 above, AUC₀₋₂₄ was about 3.8 fold greater for Group 4 (formulation B, 20/80 clyceryl monocaprylate/PEG 15 hydroxystearate), as compared to control.

### Example 8

### Optimization of Self-Emulsifying Formulations

Additional self-emulsifying formulations were tested for dilutability, maximum solubility of compound 1, and physical stability under various conditions. In particular, these formulations included addition of a co-solvent and/or a crystallization inhibiting polymer, as described below.

Data are provided for the highest achieved concentration, carrier stability at 25° C./60% RH or 40° C./75% RH for one month, as shown in Table 15. For the type of carrier, L indicates a lipophilic carrier, S indicates a surfactant carrier, and C indicates a co-solvent. Screening concentrations of compound 1 included from 25 mg/g to 400 mg/g (mg of compound 1 per g of carrier).

TABLE 15

| | | | | One month | |
|---|---|---|---|---|---|
| | | Type of | Highest | stability^{b} | |
| Carrier | Brand name | carrier | conc. (mg/g)^{a} | 25/60 | 40/75 |
| Propylene glycol | Lauroglycol ® 90 | L | <25 | n/a | |
| monolaurate, type II | (HLB 5) | | | | |
| Glyceryl monolinoleate | Maisine ® 35-1 | L | 25 | 25 | 25 |
| | (HLB 4) | | | | |
| Polyoxyl 40 hydrogenated | Cremophor ® RH 40 | S | <25 | n/a | |
| castor oil | | | | | |
| Polyoxyethylene (20) | Tween ® 80 | S | <25 | n/a | |
| sorbitan monooleate | (HLB 15) | | | | |
| Polyethylene glycol 4000 | PEG 4000 | C | 50 | 50 | <25 |
| Glyceryl monocaprylate:polyoxyl | Capmul ®:Myrj ® | L:S | 150 | <125 | <125 |
| 40 stearate | 52S (30:70) | | | | |
| Lauroyl macrogol 32 | Gelucire ® 44/14 | S | <50 | n/a | |
| glycerides | | | | | |
| Glyceryl monocaprylate | Capmul ® MCM C8 | L | 125 | <100 | <100 |
| Glyceryl monocaprylate:PEG | Capmul ®:Solutol ® | L:S | 50 | <50 | <50 |
| 15 hydroxystearate | HS 15 | | | | |
| | (20:80) | | | | |
| Diethylene glycol | Transcutol ® | C | 100 | 85 | 85 |
| monoethyl ether | | | | | |
| | N-methyl-2- | S | 400 | 200 | 150 |
| | pyrrolidone (NMP) | | | | |

n/a: not available
^{a}Highest soluble concentration, based on concentrations screened
^{b}One month stability data provided at 25° C./60% RH or 40° C./75% RH.

In addition, various formulations were tested having 5% of a co-solvent. Addition of a co-solvent could increase solubility of compound 1 and/or improve stability of the formulation. Data are provided for the highest achieved concentration and carrier stability, as shown in Table 16.

TABLE 16

| | | Type | | Highest | One month | |
|---|---|---|---|---|---|---|
| | | of | 5% Co- | conc. | stability^{b} | |
| Carrier | Brand name | carrier | solvent | (mg/g)^{a} | 25/60 | 40/75 |
| Propylene glycol | Capryol ® 90 | L | — | 85 | 75 | 85 |
| monocaprylate | | | Transcutol ® | <85 | n/a | |
| Polyoxyl 40 stearate | Myrj ® 52 | S | — | 125 | 100 | <100 |
| | | | Transcutol ® | 150 | <100 | <100 |
| Propylene glycol | Capryol ® 90: | L:S | — | 175 | <100 | <100 |
| monocaprylate: | Myrj ® 52S (20:80) | | NMP | 200 | <200 | <200 |
| polyoxyl 40 stearate | | | Transcutol ® | 200 | <175 | <175 |
| PEG 15 | Solutol ® HS15 | S | — | 65 | 50 | 50 |
| hydroxystearate | | | Transcutol ® | 75 | 75 | 75 |

| | | | | | |
|---|---|---|---|---|---|
| Propylene glycol | Capryol ® 90: | L:S | — | <75 | n/a |

| | | | | | |
|---|---|---|---|---|---|
| monocaprylate: PEG | Solutol ® HS15 | Transcutol ® | 75 | <75 | <75 |
| 15 hydroxystearate | (20:80) | | | | |

n/a: not available
^{a}Highest soluble concentration, based on concentrations screened
^{b}One month stability data provided at 25° C./60% RH or 40° C./75% RH.

From initial solubility and stability screens, Capryol®90, Myrj®52, Solutol®HS 15, or mixtures thereof, were further evaluated either with or without co-solvents Transcutol® and NMP. These data are shown in Table 17 below.

TABLE 17

| | | Highest | One week | | One month | |
|---|---|---|---|---|---|---|
| | | conc. | stability^{b} | | stability^{c} | |
| Trade name | 5% Co-solvent | (mg/g)^{a} | 25/60 | 40/75 | 25/60 | 40/75 |
| Capryol ® 90 | — | 85 | 75 | 85 | 75 | 75 |
| | Transcutol ® | <85 | n/a | n/a | n/a | n/a |
| Myrj ® 52 | — | 125 | 100 | 100 | 100 | <100 |
| | Transcutol ® | 150 | <100 | <100 | <100 | <100 |
| Capryol ® 90:Myrj ® 52S^{d} (20:80) | — | 175 | 100 | <100 | <100 | <100 |
| | NMP | 200 | <200 | <200 | <200 | <200 |
| | Transcutol ® | 200 | <175 | <175 | <175 | <175 |
| Solutol ® HS15 | — | 65 | 50 | 50 | 50 | 50 |
| | Transcutol ® | 75 | 75 | 75 | 75 | 75 |
| Capryol ® 90: Solutol ® HS15 | — | <75 | n/a | n/a | n/a | n/a |
| (20:80) | Transcutol ® | 75 | <75 | <75 | <75 | <75 |

n/a: not available
^{a}Highest soluble concentration, based on concentrations screened
^{b}One week stability data provided at 25° C./60% RH or 40° C./75% RH.
^{c}One month stability data provided at 25° C./60% RH or 40° C./75% RH.
^{d}Myrj ® 52 and Myrj ® 52S are similar polymers in different forms, where Myrj ® 52 is in the pastille form and Myrj ® 52S is in the powder form.

Various crystallization inhibiting polymers were added to binary and ternary formulations, where these crystallization inhibiting polymers included a povidone (PVP, Kollidon®30 (K30)), two hypromellose polymers (HPMCs, Methocel™ K 15M and Methocel™ K4M), hydroxypropyl cellulose (HPC), and hydroxypropylmethyl cellulose acetate succinate (HPMCAS). These combinations were prepared at 50° C. with mixing from 15 minutes and up to 24 hours, where the resultant combinations formed a solution, a clear gel, or a suspension, as shown in Table 18.

TABLE 18

| | 1% (w/w) Crystallization Inhibiting Polymers | | | | |
|---|---|---|---|---|---|
| Trade name | PVP K30 | HPMC K15M | HPMC K4M | HPC | HPMCAS |
| Capmul ® MCM C8 | solution | suspension | suspension | clear gel | solution |
| Capryol ® 90 | solution | suspension | suspension | clear gel | solution |
| Solutol ® HS15 | suspension | suspension | suspension | suspension | suspension |
| Myrj ® 52 | suspension | suspension | suspension | suspension | suspension |

Physical stability of formulations was determined by either adding the crystallization inhibiting polymer before or after adding compound 1. In one preparation method, compound 1 and the carrier(s) were dissolved together, and then the crystallization inhibiting polymer was added to the mixture. In another method, the crystallization inhibiting polymer and the carrier(s) were dissolved together, and then compound 1 was added to the mixture. Solubility was not affected by the order of incorporating the crystallization inhibiting polymer, where data for Capryol® 90 and Myrj® 52 are provided below in Table 19.

TABLE 19

| | | | | | | | | | | 1% Polymer + Excipient first, | | | | |
|---|---|---|---|---|---|---|---|---|---|---|---|---|---|---|
| | | | | API + Excipient dissolved first, then | | | | | | then API added | | | | |
| | No Polymer | | | 1% Polymer added | | | | | | Highest conc. | | | | |
| | Highest | One week | | Highest conc. (mg/g)^{a} | | | | One week | | (mg/g)^{a} | | | One week | |
| Trade | conc. | stability^{b} | | HPMC | HPMC | | PVP | stability^{b} | | HPMC | | PVP | stability^{b} | |
| name | (mg/g)^{a} | 25/60 | 40/75 | K15M | K4M | HPMCAS | K30 | 25/60 | 40/75 | K15M | HPMCAS | K30 | 25/60 | 40/75 |

| | | | | | | | |
|---|---|---|---|---|---|---|---|
| Capryol ® | 85 | 75 | 85 | 85 | 85 | 85 | 85 |
| 90 | | | | | | | |
| Myrj ® 52 | 125 | 100 | 100 | 125 | <100 | 125 | 100 |
| | | | | | | | (HPMCAS)^{b} |

^{a}Highest souble concentration, based on concentrations screened
^{b}One week stability data provided at 25° C./60% RH or 40° C./75% RH.
^{c}100 mg/g formulation with HPMCAS was stable, but the other formulations were unstable.

Further analyses showed that Capmul®:Solutol®HS 15 (20:80) systems with 1% (w/w) PVP K30 or HPMCAS were physically stable for one month at a concentration of 50 mg/g (mg of compound 1 per g of carrier). Also, Capryol®90 systems with 1% (w/w) PVPK30, HPMCAS, or HPMC K15 were physically stable for one month at a concentration of 85 mg/g (mg of compound 1 per g of carrier). For these systems, the crystallization inhibiting polymer and carrier(s) were first mixed together, and then compound 1 was added. Increasing polymer concentration up to 5% (w/w) did not greatly improve stability for Myrj® 52 formulations at one month. These data are provided in Table 20.

TABLE 20

| | | HPMC K15M | | HPMCAS | | PVP K30 | |
|---|---|---|---|---|---|---|---|
| | % (w/w) of | Highest | | Highest | | Highest | |
| | crystallization | soluble | Stable | soluble | Stable | soluble | Stable |
| | inhibiting | conc. | conc. | conc. | conc. | conc. | conc. |
| Trade name | polymer | (mg/g) | (mg/g)^{a} | (mg/g) | (mg/g)^{a} | (mg/g) | (mg/g)^{a} |
| Capmul ® MCM | 1% | <125^{b} | n/a | <125 | n/a | <125 | n/a |
| C8 | | | | | | | |
| Capmul ®: | 1% | 50 | <50/50 | 50 | 50/50 | 50 | 50/50 |
| Solutol ® HS 15 | | | | | | | |
| (20:80) | | | | | | | |
| Capryol ® 90 | 1% | 85 | 85 | 85 | 85 | 85 | 85 |
| Myrj ® 52 | 1% | 125 | <100/<100 | 100 | <100/<100 | 125 | <100/<100 |
| | 2.5% | 100 | 100/<100 | n/d | n/d | 125 | 125/<100 |
| | 5% | 100 | 100/<100 | n/d | n/d | 125 | 125/<100 |
| Capryol ® 90: | 1% | 125 | n/a^{c} | 175 | <125/<125 | 150 | <125/<125 |
| Myrj ® 52S | | | | | | | |
| (20:80) | | | | | | | |
| Capryol ® 90: | 1% | 200 | <200 | 200 | <200 | 200 | <200 |
| Myrj ® 52S | | | | | | | |
| (20:80) + 5% | | | | | | | |
| NMP | | | | | | | |

n/a: not available;
n/d: not determined
^{a}One month stability data provided at 25° C./60% RH and at 40° C./75% RH.
^{b}Highest soluble concentration with HPMC K4M was also <125 mg/g for Capmul ® MCM C8.
^{c}Stable at concentration of 125 mg/g for one week at 25° C./60% RH and 40° C./75% RH.

Overall, five systems containing crystallization inhibiting polymers were physically stable at one month at 25° C./60% RH and 40° C./75% RH. These five systems included Capmul®:Solutol® HS 15 (20:80) with 1% PVPK30 at 50 mg/g; Capmul®:Solutol® HS 15 (20:80) 1% HPMCAS at 50 mg/g; Capryol® 90 with 1% PVPK30 at 85 mg/g; Capryol® 90 with 1% HPMC-AS at 85 mg/g; and Capryol® 90 with 1% HPMC K15 at 85 mg/g. In addition, co-solvents, like Transcutol®, led to higher solubility of compound 1 in Solutol® HS 15 and Capryol®/Myrj® systems. Thus, use of any of the crystallization inhibiting polymers and/or co-solvent carriers described herein would be helpful to optimize stability and/or solubility of the present formulations.

### Example 9

### Phase 1 (Part 1) Study in Healthy Human Subjects

The pharmacokinetics of a self-emulsifying formulation can be studied in healthy, human subjects using an oral dosage of compound 1. Compound 1 can be provided in any useful self-emulsifying formulation, as described herein.

A single center, open label, crossover design study of an oral test formulation of compound 1 can be conducted to determine the bioavailability under fasted and fed conditions. Subjects can be healthy male and female volunteers. All subjects can be randomized to the formulation and can take the formulation in the fasted and fed states in a crossover manner. The study can include, for example, an up to 21 day screening period, two 4 day inpatient clinic stays, and 35 days of outpatient follow up period.

Subjects can be screened for study eligibility. After completing the screening period, subjects can report to the clinical testing facility on Day −1; undergo repeat selected safety assessments (e.g., hematology, clinical chemistry, urinalysis, drug and alcohol screen), clinical assessments (e.g., physical examination, vital signs), and a serum β-hCG pregnancy test (women only) to confirm their continued eligibility for the study; and can be admitted to the clinical testing facility.

On Day −1, the subjects for Part I of the study can be randomized to the fed state or fasted state, according to a computer-generated randomization code, in the order in which they qualify for randomization. Subjects can receive the formulation in the fasted state (defined as no food consumption for about 10 hours and no water for about 2 hours prior to oral dosing) on Period 1/Day 1. The subjects who are to receive the formulation in the fed state can consume a high fat meal about 30 minutes prior to oral dosing and must completely consume the meal before taking the formulation. Vital signs can be measured, a 12-lead ECG can be obtained, and a PK blood sample can be obtained immediately prior to dosing. Subjects then can take the formulation orally. Blood samples for PK determinations can be taken at 0.5, 1, 1.25, 1.5, 1.75, 2, 2.5, 3, 4, 5, 6, 8, 10, 12, and 16 hours post oral dose; vital signs and 12-lead ECGs can be obtained at 3, 4, and 5 hours post-dose; and any concomitant medications and adverse events (AE) can be recorded.

Blood samples for PK determinations can be obtained at 24 and 36 hours post-dose on Period 1/Day 2, at 48 and 60 hours post-dose on Period 1/Day 3, and at 72 hours post-dose on Period 1/Day 4. Vital sign measurements can be obtained immediately prior to the nominal PK sampling time point. Any concomitant medications and AEs can be also recorded on Period 1/Days 2, 3, and 4. In addition, a physical examination can be performed and fasting blood and urine samples for safety laboratories (hematology, clinical chemistry, and urinalysis), and a 12-lead ECG can be obtained on Period 1/Day 4. After completion of all of the Period 1/Day 4 assessments, the subjects can be discharged from the clinical testing facility.

Subjects can report back to the clinical testing facility on the mornings of Period 1/Days 5 and 6 for collection of PK blood samples at 96 hours (Period 1/Day 5) and 120 hours (Period 1/Day 6) post-dose. At each visit, vital signs can be measured, and concomitant medications and AEs can be recorded.

Subjects can return to the clinic in the fasted state on Period 2/Day −1 and can be admitted to the clinical testing facility at a time adequate to allow for the conduct and processing of the clinical and safety assessments that are required prior to the start of the second study period. The procedures that were performed on Day −1 can be performed to confirm each subject's continued eligibility to participate in the study. Subjects who continue to meet the eligibility criteria can be admitted to the clinical testing facility.

Subjects who received the formulation in the fed state in Period 1 can receive the formulation in the fasted state in Period 2 (defined as no food consumption for 10 hours and no water for 2 hours preceding oral dosing). Subjects who received the formulation in the fasted state in Period 1 can receive the formulation in the fed state in Period 2. All subjects can have a PK blood sample taken at 168 hours after the first oral dose of study drug in Period 1; this sample, which will be drawn immediately before the subject takes the formulation on the morning of Period 2/Day 1, can be used as the pre-dose sample for Period 2. After collection of the pre-dose PK blood sample, subjects can take the single oral dose of the formulation. Blood samples for PK determinations can be taken at 0.5, 1, 1.25, 1.5, 1.75, 2, 2.5, 3, 4, 5, 6, 8, 10, 12, and 16 hours post oral dose; vital signs and 12-lead ECGs can be obtained at 3, 4, and 5 hours post-oral dose; and any concomitant medications and AEs can be recorded.

Blood samples for PK determinations can be obtained at 24 and 36 hours post-oral dose on Period 2/Day 2, at 48 and 60 hours post-dose on Period 2/Day 3, and at 72 hours post-oral dose on Period 2/Day 4. Vital sign measurements can be obtained immediately prior to the nominal PK sampling time points. Any concomitant medications and AEs can be also recorded on Period 1/Days 2, 3, and 4. In addition, a physical examination can be performed, and fasting blood and urine samples for safety laboratories (hematology, clinical chemistry, urinalysis) and a 12-lead ECG can be obtained on Period 2/Day 4. After completion of all of the Period 2/Day 4 assessments, the subjects can be discharged from the clinical testing facility.

Subjects can return to the clinical testing facility in the fasted state for an end of study (EOS) visit on Period 2/Day 36 or at the time of premature termination from the study. A fasting blood sample for PK determinations (840 hours post-oral dosing) and fasting blood and urine samples for safety laboratory determinations can be obtained, vital signs can be measured, and a physical examination can be performed. A pregnancy test (β-hCG) can be performed for female subjects. Any concomitant medications and AEs can be recorded.

Upon recordal of data after the study, various pharmacokinetic (PK) parameters can be determined, including AUC_{0-∞}, Cₘₐₓ, and Tₘₐₓ values that can be optionally corrected for the carry-over effect between the Period 1 and Period 2. Administration of the formulation can produce enhanced PK parameters, such as a reduced coefficient of variation in AUC_{0-∞}, and/or Cₘₐₓ for fasted and/or fed subjects as compared to control (e.g., a micronized formulation with compound 1 as an HCl salt or a formulation with compound 1 in 0.5% Tween® 80 in 0.5% carboxy methylcellulose (CMC)), a coefficient of variation in AUC_{∞} or Cₘₐₓ of less than about 60% in fed and fasted subjects, a coefficient of variation in AUC_{∞} or Cₘₐₓ of less than about 65% in fed or fasted subjects, a ratio of the mean bioavailability for fed subjects to the mean bioavailability for fasted subjects of from about 1.0 to about 2.0, and/or a mean bioavailability great than about 20%.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents and patent applications are herein incorporated by reference in their entirety to the same extent as if each individual publication, patent or patent application was specifically and individually indicated to be incorporated by reference in its entirety.

What is claimed is:

1. A pharmaceutical composition in unit dosage form for oral administration, said composition comprising from about 20 mg to about 250 mg of compound 1 and a pharmaceutically acceptable carrier,
wherein, following administration of said pharmaceutical composition to subjects, the ratio of the mean bioavailability for fed subjects to the mean bioavailability for fasted subjects is from about 1.0 to about 2.0, and
wherein said composition is self-emulsifying.

2. A pharmaceutical composition in unit dosage form for oral administration, said composition comprising from about 20 mg to about 250 mg of compound 1 and a pharmaceutically acceptable carrier, wherein said composition is self-emulsifying.

3. The pharmaceutical composition of claim 2, wherein said composition comprises of from about 2% to about 10% (w/w) of said compound 1.

4. The pharmaceutical composition of claim 1, wherein the percentage loading of said compound 1 is of from about 0.1% to about 60% (w/w).

5. The pharmaceutical composition of claim 2, wherein said carrier comprises a lipophilic carrier and optionally a surfactant carrier.

6. The pharmaceutical composition of claim 1, wherein said unit dosage form comprises from about 20 mg to about 100 mg of compound 1.

7. The pharmaceutical composition of claim 2, wherein, following administration of said pharmaceutical composition to fasted subjects, the mean bioavailability is greater than about 20%.

8. The pharmaceutical composition of claim 2, wherein administration of said pharmaceutical composition to fed and fasted subjects produces a coefficient of variation in Cₘₐₓ of less than about 60%.

9. The pharmaceutical composition of claim 2, wherein administration of said pharmaceutical composition to fasted subjects produces a coefficient of variation in Cₘₐₓ of less than about 65%.

10. The pharmaceutical composition of claim 2, wherein administration of said pharmaceutical composition to fed subjects produces a coefficient of variation in Cₘₐₓ of less than about 65%.

11. The pharmaceutical composition of claim 2, wherein administration of said pharmaceutical composition to fed and fasted patients produces a coefficient of variation in AUC_{∞} of less than about 60%.

12. The pharmaceutical composition of claim 2, wherein administration of said pharmaceutical composition to fasted subjects produces a coefficient of variation in AUC_{∞} of less than about 65%.

13. The pharmaceutical composition of claim 2, wherein administration of said pharmaceutical composition to fed subjects produces a coefficient of variation in AUC_{∞} of less than about 65%.

14. The pharmaceutical composition of claim 1, wherein said carrier comprises one or more of a lipophilic carrier, a surfactant carrier, and optionally a co-solvent carrier.

15. The pharmaceutical composition of claim 14, wherein said lipophilic carrier has a hydrophobic-lipophilic balance of from 2 to 10 and said surfactant carrier has a hydrophobic-lipophilic balance of from 10 to 20.

16. The pharmaceutical composition of claim 15, wherein said carrier comprises of from about 15% to about 50% (w/w) of said lipophilic carrier and of from about 40% to about 80% (w/w) of said surfactant carrier.

17. The pharmaceutical composition of claim 14, wherein said carrier comprises a lipophilic carrier selected from the group consisting of a glyceryl ester of one or more fatty acids, a propylene glycol ester, an ethylene glycol ester, and a polyglyceryl ester.

18. The pharmaceutical composition of claim 17, wherein said lipophilic carrier is a glyceryl ester of one or more fatty acids having the formula 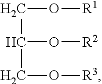
wherein R¹, R², and R³ are, independently, H or C(O)—X; each X is, independently, optionally substituted C₄₋₂₄ alkyl or optionally substituted C₄₋₂₄ alkenyl; and at least one of R¹, R², or R³ is C(O)—X.

19. The pharmaceutical composition of claim 18, wherein said glyceryl ester of one or more fatty acids is one or more of glyceryl monocaprylate, glyceryl dicaprylate, glyceryl tricaprylate, glyceryl monocaprate, glyceryl dicaprate, or glyceryl tricaprate, or a mixture thereof.

20. The pharmaceutical composition of claim 17, wherein said lipophilic carrier is a propylene glycol ester having the formula 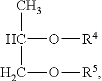
wherein R⁴ and R⁵ are, independently, H or C(O)—Y; each Y is, independently, optionally substituted C₄₋₂₄ alkyl or optionally substituted C₄₋₂₄ alkenyl; and at least one of R⁴ and R⁵ is C(O)—Y.

21. The pharmaceutical composition of claim 20, wherein said propylene glycol ester is one or more of propylene glycol monocaprylate, propylene glycol ester dicaprylate, propylene glycol monocaprate, or propylene glycol dicaprate, or a mixture thereof.

22. The pharmaceutical composition of claim 14, wherein said carrier comprises a surfactant carrier selected from the group consisting of a polyethoxylated ester of one or more fatty acids, a polyethoxylated alkyl ether, a polyethoxylated glyceryl ester, a polyoxyethylene glyceryl ester of one or more fatty acids, a sorbitan ester, a polyethoxylated sorbitan ester, and an ethoxylated propoxylated block copolymer.

23. The pharmaceutical composition of claim 22, wherein said surfactant carrier is a polyethoxylated ester of one or more fatty acids having the formula R⁶—C(O)O—(CH₂CH₂O)ₚ—R⁷,
wherein R⁶ and R⁷ are, independently, H, optionally substituted C₁₂₋₂₄ alkyl, or optionally substituted C₁₂₋₂₄ alkenyl; p is an integer of from 5 to 50; and at least one of R⁶ or R⁷ is an optionally substituted C₁₂₋₂₄ alkyl or optionally substituted C₁₂₋₂₄ alkenyl.

24. The pharmaceutical composition of claim 23, wherein said polyethoxylated ester of one or more fatty acids is selected from the group consisting of polyoxyl 40 stearate, polyoxyl 8 stearate, and PEG 15 hydroxystearate.

25. The pharmaceutical composition of claim 22, wherein said surfactant carrier is a polyethoxylated alkyl ether having the formula R⁸—O—(CH₂CH₂O)_{q}—R⁹,
wherein R⁸ and R⁹ are, independently, H, optionally substituted C₁₂₋₂₄ alkyl, or optionally substituted C₁₂₋₂₄ alkenyl; q is an integer of from 5 to 50; and at least one of R⁸ or R⁹ is an optionally substituted C₁₂₋₂₄ alkyl or optionally substituted C₁₂₋₂₄ alkenyl.

26. The pharmaceutical composition of claim 25, wherein said polyethoxylated alkyl ether is selected from the group consisting of polyoxyl 10 oleoyl ether and PEG 25 cetostearyl ether.

27. The pharmaceutical composition of claim 14, wherein said carrier comprises a co-solvent carrier selected from the group consisting of ethanol, glycerol, propylene glycol, polyethylene glycol, propylene carbonate, diethylene glycol monoethyl ether, glycofurol, and N-methyl-2-pyrrolidone.

28. The pharmaceutical composition of claim 1, wherein said carrier comprises a mixture of glyceryl monocaprylate and PEG 10 oleoyl ether.

29. The pharmaceutical composition of claim 1, wherein said carrier comprises a mixture of glyceryl monocaprylate and PEG 15 hydroxystearate.

30. The pharmaceutical composition of claim 1, wherein said carrier comprises a mixture of glyceryl monocaprylate and polyoxyl 40 stearate.

31. The pharmaceutical composition of claim 1, wherein said carrier comprises glyceryl monocaprylate.

32. The pharmaceutical composition of claim 1, wherein said carrier comprises a mixture of propylene glycol monocaprylate and polyoxyl 40 stearate.

33. The pharmaceutical composition of claim 1, wherein said carrier comprises a mixture of propylene glycol monocaprylate and PEG 10 oleoyl ether.

34. The pharmaceutical composition of claim 1, wherein said carrier comprises propylene glycol monocaprylate.

35. The pharmaceutical composition of claim 1, further comprising of from about 0.5% to about 5% (w/w) of a crystallization inhibiting carrier selected from the group consisting of cellulose acetate phthalate (CAP), methylcellulose acetate phthalate, hydroxypropyl cellulose (HPC), hydroxypropylmethyl cellulose (HPMC), hydroxypropylmethyl cellulose acetate, hydroxypropylmethyl cellulose acetate succinate (HPMCAS), a polyvinyl pyrrolidone (PVP), a polyvinyl acetate (PVA), and a copolymer of a polyvinyl pyrrolidone and a polyvinyl acetate (PVP-PA).

36. The pharmaceutical composition of claim 1, wherein said carrier, together with said compound 1, forms a stable emulsion when combined with water to form a solution that is greater than 50% (w/w) water.

37. The pharmaceutical composition of claim 1, wherein said unit dosage form comprises from about 20 mg to about 100 mg of the free base form of compound 1.

38. A method for reducing the food effect exhibited by compound 1, following administration to a subject, said method comprising administering a unit dosage form comprising the pharmaceutical composition of claim 1 to said subject.

39. A method to treat a disease or condition, said method comprising administering to a subject in need of such treatment an effective amount of the pharmaceutical composition of claim 1.

40. The method of claim 39, wherein said subject is a fasted subject.

41. The method of claim 39, wherein said subject is a fed subject.

42. The method of claim 39, wherein said condition is pain, epilepsy, Parkinson's disease, a mood disorder, psychosis, tinnitus, amyotrophic lateral sclerosis, glaucoma, ischaemia, a spasticity disorder, obsessive compulsive disorder, restless leg syndrome, or Tourette syndrome.

43. The method of claim 42, wherein said condition is pain, epilepsy, Parkinson's disease, a mood disorder, psychosis, or tinnitus.

44. The method of claim 43, wherein said psychosis is schizophrenia.

45. The method of claim 39, wherein said condition is pain or epilepsy.

46. The method of claim 45, wherein said pain is inflammatory pain or neuropathic pain.

47. The method of claim 46, wherein said inflammatory pain is caused by rheumatoid arthritis, juvenile idiopathic arthritis, ankylosing spondylitis, psoriatic arthritis, inflammatory bowel disease, primary dysmenorrhea, or endometriosis.

48. The method of claim 45, wherein said pain is chronic pain.

49. The method of claim 48, wherein said chronic pain is peripheral neuropathic pain, central neuropathic pain, musculoskeletal pain, headache, visceral pain, or mixed pain.

50. The method of claim 49, wherein
said peripheral neuropathic pain is post-herpetic neuralgia, diabetic neuropathic pain, neuropathic cancer pain, HIV-associated neuropathy, erythromelalgia, failed back-surgery syndrome, trigeminal neuralgia, or phantom limb pain;
said central neuropathic pain is multiple sclerosis related pain, Parkinson disease related pain, post-stroke pain, post-traumatic spinal cord injury pain, lumbosacral radiculopathy, cervical radiculopathy, brachial radiculopathy, or pain in dementia;
said musculoskeletal pain is osteoarthritic pain or fibromyalgia syndrome;
said headache is migraine, cluster headache, tension headache syndrome, facial pain, or headache caused by other diseases;
said visceral pain is interstitial cystitis, irritable bowel syndrome, or chronic pelvic pain syndrome; or
said mixed pain is lower back pain, neck and shoulder pain, burning mouth syndrome, or complex regional pain syndrome.

51. The method of claim 50, wherein said headache is migraine.

52. The method of claim 45, wherein said pain is acute pain.

53. The method of claim 52, wherein said acute pain is nociceptive pain or post-operative pain.

54. The method of claim 53, wherein said acute pain is post-operative pain.

55. A method to treat a disease or condition modulated by ion channel activity, said method comprising administering to a subject in need of such treatment an effective amount of the pharmaceutical composition of claim 1.

56. A method of inhibiting an ion channel, said method comprising contacting a cell with the pharmaceutical composition of claim 1.

57. The method of claim 55, wherein said ion channel is a calcium channel or a sodium channel.

58. The method of claim 38, wherein said unit dosage form is administered to achieve a daily amount of up to about 1,600 mg of compound 1.

59. The method of claim 58, wherein said daily amount is up to about 400 mg of compound 1.

60. A method of preparing a self-emulsifying pharmaceutical composition of claim 1, said method comprising:
preparing a solution comprising compound 1 and one or more of a lipophilic carrier or a surfactant carrier;
heating said solution to the solubilization temperature for said lipophilic carrier or said surfactant carrier; and
mixing said solution to form said self-emulsifying pharmaceutical composition.

61. The method of claim 60, wherein said method further comprises:
cooling said solution; and
filling said unit dosage form with said solution.

62. The method of claim 60, wherein said solution further comprises a crystallization inhibiting carrier.

63. The method of claim 60, wherein said solution comprises a lipophilic carrier and said lipophilic carrier is a glyceryl ester of one or more fatty acids having the formula 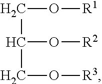
wherein R¹, R², and R³ are, independently, H or C(O)—X; each X is, independently, optionally substituted C₄₋₂₄ alkyl or optionally substituted C₄₋₂₄ alkenyl; and at least one of R¹, R², or R³ is C(O)—X.

64. The method of claim 60, wherein said solution comprises a lipophilic carrier and said lipophilic carrier is a propylene glycol ester having the formula 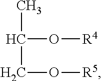
wherein R⁴ and R⁵ are, independently, H or C(O)—Y; each Y is, independently, optionally substituted C₄₋₂₄ alkyl or optionally substituted C₄₋₂₄ alkenyl; and at least one of R⁴ and R⁵ is C(O)—Y.

65. The method of claim 60, wherein said solution comprises a surfactant carrier and said surfactant carrier is a polyethoxylated ester of one or more fatty acids having the formula R⁶—C(O)O—(CH₂CH₂O)ₚ—R⁷,
wherein R⁶ and R⁷ are, independently, H, optionally substituted C₁₂₋₂₄ alkyl, or optionally substituted C₁₂₋₂₄ alkenyl; p is an integer of from 5 to 50; and at least one of R⁶ or R⁷ is an optionally substituted C₁₂₋₂₄ alkyl or optionally substituted C₁₂₋₂₄ alkenyl.

66. The method of claim 60, wherein said solution comprises a surfactant carrier and said surfactant carrier is a polyethoxylated alkyl ether having the formula R⁸—O—(CH₂CH₂O)_{q}—R⁹,
wherein R⁸ and R⁹ are, independently, H, optionally substituted C₁₂₋₂₄ alkyl, or optionally substituted C₁₂₋₂₄ alkenyl; q is an integer of from 5 to 50; and at least one of R⁸ or R⁹ is an optionally substituted C₁₂₋₂₄ alkyl or optionally substituted C₁₂₋₂₄ alkenyl.

67. The method of claim 60, wherein said solution comprises a mixture of glyceryl monocaprylate and PEG 10 oleoyl ether.

68. The method of claim 60, wherein said solution comprises a mixture of glyceryl monocaprylate and PEG 15 hydroxystearate.

69. The method of claim 60, wherein said solution comprises a mixture of glyceryl monocaprylate and polyoxyl 40 stearate.

70. The method of claim 60, wherein said solution comprises glyceryl monocaprylate.

71. The method of claim 60, wherein said solution comprises a mixture of propylene glycol monocaprylate and polyoxyl 40 stearate.

72. The method of claim 60, wherein said solution comprises a mixture of propylene glycol monocaprylate and PEG 10 oleoyl ether.

73. The method of claim 60, wherein said solution comprises propylene glycol monocaprylate.

74. The method of claim 60, wherein said solution further comprises a co-solvent carrier selected from the group consisting of ethanol, glycerol, propylene glycol, polyethylene glycol, propylene carbonate, diethylene glycol monoethyl ether, and glycofurol.

75. The method of claim 60, wherein said unit dosage form is a hard gelatin capsule, a hard hydroxypropyl methylcellulose capsule, or a soft gelatin capsule.

76. The method of claim 60, wherein said unit dosage form comprises from about 20 mg to about 100 mg of compound 1.
